(19) United States
(12) Patent Application Publication
(10) Pub. No.: US 2005/0197783 A1
(43) Pub. Date: Sep. 8, 2005

(54) Methods and systems for extension, exploration, refinement, and analysis of biological networks

(76) Inventors: Allan J. Kuchinsky, San Francisco, CA (US); Aditya Vailaya, Santa Clara, CA (US); Annette Adler, Palo Alto, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/794,341

(22) Filed: Mar. 4, 2004

(51) Int. Cl. G06F 19/00; G01N 33/48; G01N 33/50

(52) U.S. Cl. 702/19; 707/1

(57) ABSTRACT

Systems, methods and recordable media for facilitating user-guidance of computational analysis and knowledge extraction tools for use with biological data for disambiguation and determination of causation in representations of the data. Systems, methods, tools and computer readable media for user-guided expansion of biological networks and manipulations of the same. Tools for qualitative simulation of network models, for identifying interactions between multiple networks and form maintaining knowledge about multiple alternative biological networks. Combinations of portions of networks may be constructed to provide optimal networks. Networks may be evaluated against experimental data and networks of interest may be identified based on experimental data.

### FIELD OF THE INVENTION

The present invention pertains to manipulation of biological data. More particularly, the present invention pertains to systems, methods and recordable media for interactively importing, creating and/or manipulating biological diagrams, which may be based on a variety of data sources.

### BACKGROUND OF THE INVENTION

The discovery of medicines and treatments for life-threatening diseases is often a process of piecing together a detailed understanding of the molecular basis of disease, a process of putting together and articulating the story of how genes and proteins interact with each other in biological networks. By understanding the structure and behavior of biological networks, i.e. the elements of the networks and the complex sets of interactions between them, biomedical researchers can identify intervention points for drugs and therapeutics, limit adverse side-effects of treatments, and infer predisposition to disease.

Molecular biologists working in this area need to assimilate knowledge from a dramatically increasing amount and diversity of biological data. The advent of high-throughput experimental technologies for molecular biology have resulted in an explosion of data and a rapidly increasing variety of biological measurement data types. Examples of such biological measurement types include gene expression from DNA microarray or Quantitative Polymerase Chain Reaction (PCR) experiments, protein identification from mass spectrometry or gel electrophoresis, cell localization information from flow cytometry, phenotype information from clinical data or knockout experiments, genotype information from association studies and DNA microarray experiments, etc. This data is rapidly changing; new technologies frequently generate new types of data. In addition to data from their own experiments, biologists also utilize a rich body of available information from Internet-based sources, e.g. genomic, proteomic, and pathway databases, and from the scientific literature.

Biologists may use these experimental data and numerous other sources of information to piece together interpretations and form hypotheses about biological processes. Such interpretations and hypotheses constitute higher-level models of biological activity. Such models can be the basis of communicating information to colleagues, for generating ideas for further experimentation, and for predicting biological response to a condition, treatment, or stimulus. Frequently these models take the form of biological networks and can be represented by network diagrams.

Current efforts at providing systems to generate biological network information, such as protein-protein interaction networks, via knowledge extraction, and display outputs via network diagrams, include those of Ariadne Genomics (www.ariadnegenomics.com), Apelon (www.apelon.com), BioSentients (http://www.io-informatics.com/technology.html), BioWisdom (www.biowisdom.co.uk), Cellomics CellSpace™ (http://cellspace.cellomics.com/CellSpace/default.asp), Definiens (www.definiens.de), Gene Ed/Reel Two (www.geneed.com www.reeltwo.com), Incellico (www.incellico.com), Ingenuity (www.ingenuity.com), Insightful (www.insightful.com), Iridescent (http://innovation.swmed.edu./Biocomputing/Computing.htm), Pre-BIND (http://www.binddb.org), PubGene (http://www.pubgene.com/), Virtual Genetics (www.vglab.com), and XMine (http://www.x-mine.com/). These systems rely on statistical and linguistic natural language processing to automatically pre-compute protein-protein interactions from scientific text into a database. They therefore, present a completely generated network to the user; there is no opportunity for the user to guide and/or improve the process of knowledge extraction by disambiguating and/or assigning directionality or causality.

Several computational analysis tools apply Bayesian and other machine learning methods to predict causal relationships from observational and experimental data, such as gene expression data. Examples of the use of Bayesian induction and inference methods to infer networks from measured gene expression include Nir Friedman's group's work on “Inferring Subnetworks from Perturbed Expression Profiles” (http://www.cs.huji.ac.il/˜nir/Papers/PREF.pdf) and Yoo et al. on “Discovery of Causal Relationships in a Gene Regulation Pathway from a Mixture of Experimental and Observational DNA Microarray Data” (http://www.smi.stanford.edu/proiects/helix/psb02/yoo.pdf).

As with the knowledge extraction methods, these machine learning and inference approaches present a completely generated network to the user, providing no opportunities for user guidance or improvement. Moreover, these machine learning and inference approaches characteristically are grounded in purely mathematical and statistical methods and cannot take advantage of prior biological knowledge to influence their scoring metrics.

A number of biological model (e.g., KEGG, Transfac, Transpath, SPAD, BIND, etc.) databases have been developed (both public domain and proprietary) that allow users to query and download biological models of interest. However, the user can only view these biological models after downloading them, and cannot add meaningful data or edits to a model given its static nature. Tools to import these diagrams, extract contents from them, link the extracted information to other types of data (such as experimental data, scientific text, information about concepts of interest, etc.), and use this knowledge to refine and improve the network diagram are only recently starting to be developed, and even recent developments leave needs for further extending and refining network diagrams.

### SUMMARY OF THE INVENTION

The present invention provides systems, methods and computer readable media for facilitating user-guidance of computation analysis and knowledge extraction tools, giving a user the ability to disambiguate network diagram representations of biological data, as well as to explore and determine causalities of phenomenon being studied.

Methods, systems tools and computer readable media for extending biological networks are provided. For example, at least a portion of a biological network may be provided in an interactive format representing concepts and relationships between concepts that occur in the biological network. Concepts and at least one relationship may be extracted from at least one data source provided in the interactive format, which is external to the biological network. At least one filter may be set to include at least one selected concept or relationship from the interactive format representation of the biological network, and such selected concepts and/or relationships are matched with concepts and relationships provided in the interactive format representation of the at least one external data source. The biological network may then be extended by merging concepts represented in the interactive format from the at least one data source matching concepts in the biological network.

The external data sources may be varied, and include textual data sources, experimental data sources, network diagram data sources, protein-protein interaction databases, manually constructed networks, and combinations thereof.

Multiple filters may be set, and the biological network may be extended by only those interactions which have at least one matching concept selected in each of the multiple filters.

The extended portions of a biological diagram may be identified by indicators representing where different extensions originated from, i.e., which external base the extended data originated from.

Filters may be interactively set by a user.

Stencils may be used to identify concepts and relationships within a diagram or data from a dataset that meets the requirements of the stencils according to the rules associated with that particular stencil. By applying these rules, data in an identified area of a network or dataset may be verified, or identified as containing a discrepancy, after which a user may interactively modify a representation to disambiguate it.

Methods, systems, tools and recordable media (computer readable media) are provided for extending a biological network wherein at least a portion of a biological network is provided in an interactive format representation of concepts and relationships between concepts that occur in the biological network, and additional network diagrams are constructed from concepts and relationships extracted from at least one data source external to the biological network and converted to the interactive format. At least one filter may be interactively set to include at least one selected concept existing in the biological network. The additional network diagrams are then searched to identify those additional network diagrams that contain at least one concept matching at least one selected concept. The biological network is then extended with all relationships from the additional network diagrams that are directly linked to at least one of the matching concepts, by merging them with matching concepts on the biological network and extending the directly linked relationships, including all other concepts from the additional network diagrams that are directly linked by the directly linked relationships. Further, a filter may be set to include additional levels (links or relationships and concepts) beyond those concepts which are directly linked to the matching concepts.

In an example provided using high throughput microarray data, the concepts in the biological diagram represent genes, and the relationships represent interactions between genes, At least one filter in this example is set by inputting a list of genes identified from experimental data and represented in the interactive format.

Methods, systems, tools and computer readable media are provided for interactively manipulating biological data via user guidance, to include providing a plurality of network diagrams represented in an interactive format representing concepts and relationships between concepts that occur in the network, wherein the concepts and relationships in each network diagram represent data from at least one of the data sources selected from the group consisting of textual data sources, experimental data sources, network diagram data sources, protein-protein interaction databases, manually constructed networks or any combination thereof, and at least one of the networks diagrams having been extended using data extracted from one of the data sources, Evaluation of the network diagrams may be performed by comparing the concepts and relationships among the network diagrams.

Such comparison may include displaying at least a portion of the plurality of network diagrams simultaneously in a viewer to provide a direct visual comparison of the displayed network diagrams. Further, the displayed view of the multiple diagrams may be rearranged to display those diagrams which have similarities, disparities of common concepts and relationships, nearer or adjacent to one another.

Additionally, or alternatively, evaluation may include computationally validating and displaying at least one of consistencies and inconsistencies in the network diagrams.

Further additionally or alternatively, evaluation may include overlaying data from at least one data source not represented by the network diagrams, over at least one of the network diagrams and indicating whether the overlaid data is consistent or inconsistent with the representation in the at least one network diagram that has been overlaid. Identifiers may be displayed to indicated the sources of data overlaid.

Based on evaluation results, a selection of a diagram may be made which is considered to best correspond to the overlaid data. Best correspondence may be determined by selecting that diagram which has the greatest number of consistencies, least number of inconsistencies, or best score based on a combination of numbers of consistencies and inconsistencies, for example.

Further, portions of at least two of the overlaid network diagrams may be selected for being different portions having been overlaid with consistent data. These portions may then be combined to form a new network diagram that is more consistent with the overlaid data than any of the other network diagrams.

Network diagrams may be visually differentiated as to curated and non-curated portions of the same. Examples of such visual differentiation include making the lines representing the curated portions of a different thickness than the lines representing the non-curated portions, or color-coding the curated and non-curated portions to have different colors, for example.

Further, associations in a network diagram may be visually differentiated from pathway interactions in the network diagram.

Systems, tools, methods and recordable media are provided for searching across multiple networks and identifying interesting common features among the multiple networks. For example, at least one concept may be selected, and multiple network diagrams may be searched to identify those networks having at least one occurrence of the selected concept(s). All occurrence of the selected concept(s) may be identified in all of the network diagrams that they occur in, as well as the locations where they occur.

Further, two concepts may be selected, and multiple networks may be searched to identify occurrences of the two concepts in any of the networks. All shortest paths existing between the identified concepts may also be identified and located. Further, the overall shortest path may be determined.

Other graphical analysis functions are provided, such as calculating a minimum spanning tree of a network diagram, finding a size or order of a graph representing a network, finding connectivity distributions in a network, etc.

At least one stencil may be selected as a basis upon which to search multiple network diagrams.

Methods, tools, systems and computer readable media are provided for performing simulations in network diagrams. For example, at least one biological network may be provided in an interactive format representing concepts and relationships between concepts that occur in the biological network. A value of at least one concept in the biological network may be set, from which a simulation process is propagated. The propagation is performed to extend at least one relationship downstream of the at least one value set concept. Any effects on concepts connected by the at least one relationship downstream of the at least one value set concept are then displayed.

The set value or values may be taken from experimental data corresponding to the concept or concepts for which the values are set, respectively. At least one value of at least one concept downstream of a concept having had its value set, which results from the propagation, may then be compared with a value in the experimental data corresponding to the at least one concept downstream, to validate a portion of the network, based on the experimental data or to identify a discrepancy between the portion of the network and the experimental data.

Multiple networks may be provided as interactive format representations of concepts and relationships between concepts that occur in the respective networks. The value setting process is then applied to a concept occurring in each respective network diagram, and comparisons of the type described above may be performed on corresponding downstream concepts, to identify consistency or discrepancies between the networks.

At least one set of corresponding values for at least one concept downstream of a value set concept may be compared with a value in the experimental data corresponding to the at least one concept downstream, to validate a portion of each network, based on the experimental data or to identify a discrepancy between the predicted values of the portion of the network and the values of the experimental data.

A network diagram which contains the least number of discrepancies with respect to the experimental data may be selected as the best model among the diagrams examined, for representing the experimental data.

Multiple concepts may have values set to propagate simulations in multiple portions of one or more diagrams to perform similar analyses on multiple portions of the network diagrams. From these results a determination may be made as to the best portions of diagrams examined. These best portions may then be combined to form a new network diagram that is more consistent with the experimental data than any of the other network diagrams.

The effects downstream of the propagations may be determined by rules contained by a simulation tool performing the propagation. The rules may be modular and capable of being plugged in and out of the simulation tool to tailor the simulation tool to the particular types of network diagrams and experimental data being analyzed. The effects generated by the simulation propagation may be expected values of the downstream concepts, for example.

Methods, systems, tools and computer readable media are also provided for identifying cross-talk across different networks. For example, multiple networks represented in the interactive format representing concepts and relationships between concepts that occur in the network, respectively may be provided, and a value may be set for a concept, as described above. Propagation is then performed through all downstream relationships in each respective network, and identifications are made of networks that contain downstream concepts having changed values affected by the propagation, and the locations of these concepts.

Further, subnetworks defined by the downstream concepts having changed values affected by the propagation, and their locations within the respective networks may be identified.

Alternatively, after setting the same value for the same concept in each corresponding network, each network containing downstream concepts having changed values affected by the propagation may be used as the basis for querying a database containing an additional number of networks represented in the interactive format. Networks are identified from the additional number which contain at least one of the downstream concepts affected by the propagation in at least one of the networks upon which the propagation was performed, and then these newly identified networks are propagated from the at least one identified concept through all downstream relationships in each respective identified network. Each network from the additional number containing downstream concepts having changed values affected by the propagation are then identified. Further, subnetworks defined by the downstream concepts having changed values affected by the propagation in the networks identified from the additional number of networks may be identified, as well as their locations within the respective networks.

Methods, tools, systems and computer readable media are provide for evaluating network relevance to representation of high throughput data. A set of data that are differentially expressed under different conditions may be provided and at least one network representative of the set of data may be considered to determine the number of matching data points in each network. The relevance of each network considered is then statistically determined, based on the number of data points that are in the set and also in the network, respectively.

The set of data referred to above is a subset of a larger set of high throughput data that has been determined to be more differentially expressed than the remainder of the set of high throughput data.

Statistical analysis for relevance may include Z-scoring, and a network may be considered relevant when scored with a Z-score having an absolute value of greater than about three.

Machine learning inference tools may be run iteratively and user assessment of the results of a set of iterations may be utilized to guide the operation of subsequent iterations. Thus a user may play an interactive role in optimizing results achieved by the machine learning tool used. For example, users may either explicitly or implicitly identify “good” and “bad” networks and/or network segments. Such identifications may be used as input parameters to subsequent iterations of the analysis algorithm used in a machine learning tool.

Methods, tools, systems and computer readable media for facilitating the analysis of a biological network are provided to include providing a biological network containing at least one of curated concepts and relationships, and non-curated concepts and relationships; and displaying the curated concepts and relationships in a manner differentiating the display of the non-curated concepts and relationships.

Methods of forwarding a result obtained from any of the above described methods are also covered, as are transmitting data representing a result obtained from any of the described methods to a remote location, as well as receiving a result obtained from any of the above described methods from a remote location.

These and other advantages and features of the invention will become apparent to those persons skilled in the art upon reading the details of the methods, tools, systems and computer readable media as more fully described below.

### BRIEF DESCRIPTION OF THE DRAWINGS

FIG. 1 illustrates a general overview of high throughput data analysis techniques that may be performed according to the present invention.

FIG. 2 schematically shows the components of an ALFA architecture that may be employed in the formation of ALFA objects for use according to the present invention.

FIG. 3 shows a portion of a display from an experimental viewer being used to analyze twelve microarray experiments to study a particular phenomenon or disease.

FIG. 4 is a schematic representation of a view displayed by a text viewer during the process of extracting pertinent information from a scientific journal article that has been identified from a text database.

FIG. 5A is a schematic representation of a view displayed by a network viewer displaying a network of nodes (which, in this example, represent genes) and links (which in this example, represent relations between genes).

FIG. 5B is a display of an extended network according to the present invention.

FIG. 6A schematically represents a visualization showing experimental data being overlaid on a network diagram.

FIG. 6B shows the results of propagation by a simulation tool using the network and set data shown from FIG. 6A.

FIG. 6C shows further propagation by the simulation tool using the network and set data values from FIGS. 6A-6B.

FIG. 7 is a flow chart illustrating network extending capabilities as well as some of the processes for evaluating networks that may be carried out with the present system.

FIG. 8 is a block diagram illustrating a typical computer system which may be employed in carrying out the present invention.

### DETAILED DESCRIPTION OF THE INVENTION

Before the present systems, methods and recordable media are described, it is to be understood that this invention is not limited to particular datasets, data sources, diagrams, method steps, analysis or applications described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a gene” includes a plurality of such genes and reference to “the diagram” includes reference to one or more diagrams and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

In the present application, unless a contrary intention appears, the following terms refer to the indicated characteristics.

The term “biological diagram” or “diagram”, as used herein, refers to any graphical image, stored in any type of format (e.g., GIF, JPG, TIFF, BMP, diagrams on paper or other physical format, etc.) which contains depictions of concepts found in biology. Biological diagrams include, but are not limited to, pathway diagrams, cellular networks, signal transduction pathways, regulatory pathways, metabolic pathways, protein-protein interactions, interactions between molecules, compounds, or drugs, and the like.

A “biological concept” or “concept” refers to any concept from the biological domain that can be described as one or more “nouns” according to the techniques described herein.

The term “biological network”, “network” or “network diagram” refers to a biological diagram depicting at least one relationship between at least two biological concepts.

A “curated network” is a network that has been manually verified and represents some known (or assumed known) biological process.

A “non-curated network” is a network that is inferred from automatic analyses, such as interactions and associations derived from literature and experimental data (such as Bayesian inference from microarray data, Y2H studies, etc.), or added manually based on some assumptions and hypotheses and hence is not verified. Note that a network can also be partially curated, wherein, some of the interactions (relationships) in the network are curated, but others are not.

A “relationship” or “relation” refers to any concept that can link or “relate” at least two biological concepts together. A relationship may include multiple nouns and verbs.

An “entity” or “item” is defined herein as a subject of interest that a researcher is endeavoring to learn more about, and may also be referred to as a biological concept, as belonging to that larger set. For example, an entity or item may be one or more genes, proteins, molecules, ligands, diseases, drugs or other compounds, textual or other semantic description of the foregoing, or combinations of any or all of the foregoing, but is not limited to these specific examples.

An “interaction” relates at least two entities or items. Interactions may be considered a subset of “relationships”.

An “association” between a set of concepts is defined as an indirect link between these concepts.

A “pathway interaction” is defined as one where there is a direct link between the concepts.

An “annotation” is a comment, link, or metadata about an object, entity, item, interaction, concept, relationship, diagram or a collection of these. An annotation may optionally include information about an author who created or modified the annotation, as well as timestamp information about when that creation or modification occurred.

The term “user context” refers to a collection of one or more objects, entities, items, interactions, concepts and/or relationships that describe the interests of a user when operating the present system. User context may include a set or sets of concepts and relationships.

A “database” refers to a collection of data arranged for ease and speed of search and retrieval. This term refers to an electronic database system (such as an Oracle database) that would typically be described in computer science literature. Further this term refers to other sources of biological knowledge including textual documents, biological diagrams, experimental results, handwritten notes or drawings, or a collection of these.

A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA (peptide nucleic acid) and other polynucleotides, regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

An “array” or “microarray”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably. A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice).

When one item is indicated as being “remote” from another, this is referenced that the two items are at least in different labs, offices or buildings, and may be at least one mile, ten miles, or at least one hundred miles apart.

“Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic or optical disk may carry the programming, and can be read by a suitable disk reader communicating with each processor at its corresponding station.

“May” means optionally.

A “node” as used herein, refers to an entity, which also may be referred to as a “noun” (in a local format, for example). Thus, when data is converted to a local format according to the present invention, nodes are selected as the “nouns” for the local format to build a grammar, language or Boolean logic.

A “link” as used herein, refers to a relationship or action that occurs between entities or nodes (nouns) and may also be referred to as a “verb” (in a local format, for example). Verbs are identified for use in the local format to construct a grammar, language or Boolean logic. Examples of verbs, but not limited to these, include upregulation, downregulation, inhibition, promotion, bind, cleave and status of genes, protein-protein interactions, drug actions and reactions, etc.

The term “local format” or “local formatting” refers to a common interactive format into which knowledge extracted from textual documents, biological data and biological diagrams can all be converted so that the knowledge can be interchangeably used in any and all of the types of sources mentioned. The local format may be a computing language, grammar or Boolean representation of the information which can capture the ways in which the information in the three categories are represented.

The term “ALFA object” refers to a fundamental data structure that implements the local format. ALFA primitive objects include concepts, relations, roles, nodes and networks.

A “concept” refers to a biological entity, such as a gene, protein, molecule, ligand, disease, drug or other compound, process, etc. A list of properties may be attached to a concept. Such properties may include name, aliases, sequence information, contextual information about the concept (such as state (active, inactive, post-translational modifications, etc.)), location, etc. A concept may be expressed as a node in a network diagram.

A “relation” or “relationship” is an interaction between multiple concepts. A list of properties may be attached to a relation. Such properties may include name, type (e.g., activation, inhibition, catalytic, etc.), location, etc. A relation may be expressed as a link in a network diagram.

A “node” object connects multiple relations together by connecting the roles of a common concept between different relations. If two roles of a concept are not connected, then two different node objects are created for the two roles of that concept. A node may thus act as a bridge between two or more relations.

Each concept may play a specific “role” in a relation. Currently defined roles in ALFA include upstream, downstream, mediator, container, and unknown.

A network may include a list of relations and nodes. Hierarchical structure is incorporated into ALFA via networks. A network may also be considered a concept, and, when represented as such, abstracts its list of relations and nodes to a user. For example, the relation “epinephrine inhibits glycolysis” would be represented in ALFA as epinephrine as an upstream concept and glycolysis as a downstream concept of an inhibitory relation. However, the process of glycolysis may also be represented as a set of relations, specifying the step in the anaerobic breakdown of glucose to pyruvate, yielding two molecules of ATP, and stored as a network. Therefore biological processes may be hierarchically represented through the representation of a network as a concept.

A “classifiable object” defines an ontological term. Both category and relation objects are also classifiable objects to which ontological terms may be attached.

The “base ontology” is a default ontology that is provided with the ALFA application programmer's interface (API).

All patents, patent applications and other references cited in this application, are incorporated into this application by reference except insofar as they may conflict with those of the present application (in which case the present application prevails).

Reference to a singular item, includes the possibility that there are plural of the same items present.

Methods recited herein may be carried out in any order of the recited events which is logically possible, as well as the recited order of events.

Biological networks are great repositories for information related to the current understanding of the mechanisms underlying various biological processes. Given the tremendous amounts of data being generated by current high-throughput technologies in the life sciences, there is a need for researchers to be able to identify information about entities of interest from existing biological networks, and be able to verify/validate these using proprietary experimental results in an efficient, computationally-assisted manner. Although a number of biological network databases have been developed (both public domain and proprietary) that allow users to query and download biological diagrams/networks of interest, once downloaded, they are very difficult for the user to work with. Although they can be readily viewed, the tools for editing and extending such networks, through either graphical annotations or graphical overlays, based on new knowledge and data, are extremely limited, as noted above. Further, annotation of existing networks is not supported. Often the user has a very great amount of experimental data that needs to be analyzed/compared, and manual comparison of such data with one or more models is extremely tedious to the point that it is effectively impractical to do with any amount of efficiency.

Biological networks may be dependent upon or relate to many different cellular processes, genes, and various expressions of genes with resultant variations in protein and metabolic abundance. Correlation and testing of data against these networks is becoming progressively more tedious and time-consuming, given the increasing efficiencies in the abilities and speeds of high-throughput technologies for generating gene expression, protein expression, and other data (e.g., microarrays, RT-PCR, mass spectroscopy, 2-D gels, etc.), and with the consequent increasing complexity and number of networks that describe this data. Additionally, there are many sources of textual information that describe or relate to the concepts and relationships depicted in biological networks. Organization and referencing of these textual materials with related items in biological networks has become an organizational nightmare.

The present invention provides systems, tools, methods and recordable media for expanding existing biological networks using a number of different sources of information and a number of control mechanisms via filters. Further provided are tools for evaluating the expanded networks via visualization and computational methods.

In one aspect, the present invention builds upon the advantages and capabilities provided by the network building, visualization and manipulation tools, systems and methods provided by our earlier filed, co-pending application Ser. No. 10/154,524 filed May 22, 2002 and titled “System and Method for Extracting Pre-Existing Data from Multiple Formats and Representing Data in a Common Format for Making Overlays”. Application Ser. No. 10/154,524 is hereby incorporated herein, in its entirety, by reference thereto. Information from various sources of data may be stored in a restricted grammar, referred to as the local format. This restricted grammar serves as a biological object model that can be manipulated by various tools in preparing and manipulating biological diagrams, among other functions.

Biological diagrams may be constructed, added to, or modified, based on information from a number of sources, including, but not limited to: scientific literature, experimental data, network diagrams, protein-protein interaction databases, and manually inputted information. Scientific literature includes a huge repository of the collective information of models for biological processes. Information from scientific literature that can be captured in a structured way and integrated with experimental data greatly may greatly facilitate the construction and sharing of biological models and biological networks, especially regarding unfamiliar genes and pathways. Biologists frequently use information from scientific literature to vet their working biological network models, and to amend or extend them. Methods, systems and tools for such knowledge extraction from scientific literature and use of the extracted information is described in commonly owned, co-pending application Ser. No. 10/642,376 filed Aug. 14, 2003 and titled “System, Tools and Method for Viewing Textual Documents, Extracting Knowledge Therefrom and Converting the Knowledge into Other Forms of Representation”, which is incorporated herein, in its entirety, by reference thereto. Automated text mining techniques are used to extract “nouns” (e.g. biological entities) and “verbs” (e.g. relationships) from sentences in scientific text. The resulting interpretation is then represented in the local format. The local format (structured grammar) serves as a structured way for the user to review and understand the essence of a scientific text. A model of a biological network may be generated by stitching together the set of biological entities and relationships extracted from scientific text.

Another means of generating network diagrams is through use of exploratory data analysis tools, such as those provided in co-pending, commonly owned application Ser. No. 10/403,762 filed Mar. 31, 2003 and application Ser. No. 10/688,588 filed Oct. 18, 2003, both of which are titled “Methods and System for Simultaneous Visualization and Manipulation of Multiple Data Types”, and both of which are incorporated herein, in their entireties, by reference thereto. These disclosures provide efficient methods to explore and visually identify patterns in the data, and are ideally suited for finding correlations in gene expression data between sets of genes and various experimental conditions. Known interactions between these sets of genes and the experimental conditions can be identified using the method described in application Ser. No. 10/154,524. The concepts identified and their relationships may then be represented in the local format and used interactively to make, modify or interact with (such as by overlays, for example) biological diagrams.

Existing biological diagrams may also be used as a source of biological information in performing the functions of the present invention. Currently, most biological diagrams/networks exist as static images (GIF, JPEG, Bitmap, etc.) and are not usually machine-readable. Automated analysis of these networks to extract the underlying knowledge suffers from the same limitations as automated text mining methods. Co-pending, commonly owned application Ser. No. 10/155,675 filed May 22, 2002 and titled “System and Methods for Extracting Semantics from Images” describes methods for extracting knowledge from such static biological networks and converting them to a structured representation (i.e., local format). Biological information of this sort may be stored in public and private databases, and may exist as images in books and journal articles, or sketches on paper. Application Ser. No. 10/155,675 is incorporated herein, in its entirety, by reference thereto.

Yet another source of biological information useful for construction and manipulation of biological networks according to the present invention includes protein-protein interaction databases, such as BIND, DIP, etc. These databases are mainly constructed based on manual extraction from scientific literature or experimentally from Y2H (yeast-2-hybrid) studies. A network diagram can be constructed from the transitive closure of the results (after converting the results into the local format) returned by querying these databases for interactions between pairs of genes or proteins.

Still further, networks may be drawn by a user using a tool, such as described in co-pending, commonly owned application Ser. No. 10/641,492 filed Aug. 14, 2003 and titled “Method and System for Importing, Creating and/or Manipulating Biological Diagrams”, which is incorporated herein, in its entirety, by reference thereto. Application Ser. No. 10/641,492 discloses conversion of a constructed network to the local format to provide interactive capabilities with the network.

In addition to constructing networks from any and all of the above sources of information, including any combination of these sources, these constructed networks or existing networks that have been processed into the local format may be further expanded by combining one or more interactive objects, such as ALFA objects, with the network in locations where the interactive object has a node or other data representation that matches one on the diagram. Further, user interactivity is provided by the ability of the system to discriminately expand a network based upon only selected features from the data used to expand the network with. Such selected features may be defined by filters set up according to the selections made by a user. Alternatively, filters may be preset for a user, as an option.

A filter may be defined to add to a network diagram only those interactions (generated from any of the sources of data, or combinations thereof, described above), which pass the given filter. For example, a filter may specify elements selected from the network diagram to be expanded. These elements may be only certain nodes or may include nodes and links that, for example may describe a particular interaction among genes. A filter may be set so as to expand a given network diagram with only those interactions from any number of network diagrams (constructed from any of the data sources or combinations thereof, described above), which have at least one concept from the given network.

A parameter referred to as “concepts per relation” may be set to filter out from the set of interactions those interactions with a greater number of concepts than that specified by the “concepts per relation” parameter.

A filter may also be provided with a parameter, defined as the level, which may be set with an integer greater than or equal to 1. For example, consider the situation where there is a first network, N1, that has the an interaction wherein concept A promotes concept B, and another network, N2, that has the following interactions: concept C promotes concept D, concept D promotes concept E, and concept E promotes concept A, and wherein a filter file, F, is set such that it has concept A in it. By setting the level L of filter file F to 1, and extending network N1 with network N2, using the filter F, the extension operation results in the addition of the relation “concept E promotes concept A” to network N1 (i.e., concept E in network N2 is directly connected to concept A, which is in network N1 and also in the filter file F). If, however, level L of filter F is set to 2, then the same extension procedure adds two interactions (relations) from network N2 into network N1. Specifically, the interactions “concept E promotes concept A” and “concept D promotes concept E” are both added to network N1, because, by setting L=2, the extension is now performed such that any node that is added is connected to a node in the filter within L steps. In the above example, concept D is connected to concept E in 1 step (level), and concept E is connected to concept A in another step (level), making concept D connected to concept A in 2 steps (levels). Of course, this is only an example, as more than two levels can be extended in an extension operation by setting L to an integer value greater than 2.

Arbitrary concepts may be entered into a filter by a user. For example, a user may set up a filter to expand a given network with all interactions from any number of network diagrams (constructed from any of the data sources described above, or combinations thereof), which have at least one of the concepts from the a list of concepts inputted to the filter by the user.

As another example, gene lists (such as up-regulated genes from experimental data), may be used to expand a network. For example, the filter may be constructed to expand a network diagram with only those interactions from any number of other network diagrams (constructed from any of the data sources described above, or combinations thereof.), which have at least one concept matching a gene in the up-regulated list of genes provided by the user.

Further, protein abundance lists (e.g., from SpectrumMill, available from Agilent Technologies, Inc., Palo Alto, Calif.) may be used as filter settings to expand a network with only those interactions from any number of network diagrams (constructed from any of the data sources described above, or combinations thereof.), which have at least one concept matching a protein in a list of proteins inputted to the filter by the user.

Multiple filters may also be set for expanding a network. For example, two different filters can be set, such that a network is expanded by only those interactions from any number of network diagrams (constructed from any of the data sources described above, or combinations thereof.), which have at least one gene from both the filters. A typical use case (though not restricted to only this use case) while analyzing gene expression data may include identification of genes that are differentially regulated under different conditions (say, diseased vs. normal state of a biological process). The above-described principles make it possible to expand diagrams, including existing diagrams found in databases such as KEGG, and a number of other publicly available databases. As noted, such expansion can be based upon other sources of information (such as scientific literature or information in databases, such as BIND), with specific interactions representing differentially regulated genes, for example. In this example, two filters are set (one for the selected initial approximate model as found in KEGG, and the other for the up-regulated set of genes identified in diseased tissue experimental data) for expanding the initial network diagram. Similarly, a down-regulated set of genes in the diseased state (up-regulated in the normal state) can be used to expand the same initial network (as found in KEGG) to model the biological process under the normal condition. Thus, existing network models can be expanded using multiple filters to generate models of the underlying biological process under different conditions.

The use of filters to expand biological networks can be very useful in manual modification and extension of simple models into complex models over time, without inundating the user with a plethora of data all at once.

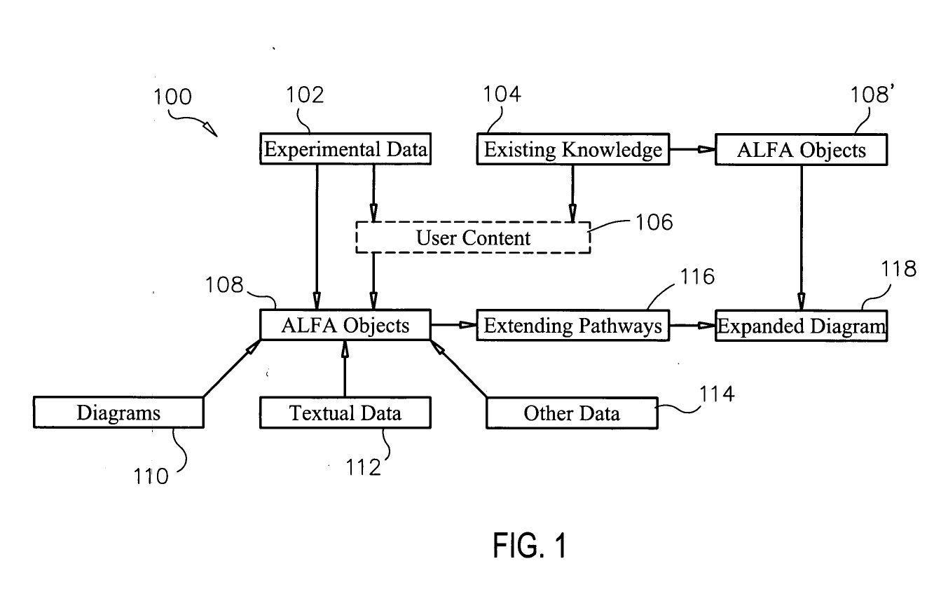

FIG. 1 illustrates a general overview 100 of high throughput data analysis techniques that may be performed according to the present invention. High throughput experimental data 102, such as microarray gene expression data, as a non-limiting example, may be considered. A simple example of such high throughput microarray data is a microarray measuring cancer tissue and normal tissue, where the experimenter wishes to see how the genes are differentially regulated in comparison between the tissues. A problem posed to the researcher/experimenter, is that the genes that differentially expressed among the two conditions (e.g., normal tissue versus cancer tissue) may number on the order of thousands. Typically the researcher will not have knowledge of all of these genes, but may only have specific knowledge about a few genes which have been reported in the literature to have particular relevance to the phenomenon that the researcher is studying, or which they have done prior research on.

The present systems therefore endeavor to put the differentiated genes into biological context, to facilitate a directed approach to the researcher's study of the data most likely to yield fruitful results. For example, an exploratory data analysis tool such as described in application Ser. No. 10/403,762 or application Ser. No. 10/688,588 may be used to identify a set of genes which are differentially regulated from dataset 102, or differential protein levels if dataset is a set of protein data. It is reiterated here, for emphasis, that the experimental data that may be used as an input for the present invention is not limited to microarray data or protein data, but may be any high throughput biological data. For example, mass spectrograph data, such as may be provided by a product known as SpectrumMill, available from Agilent Technologies, Inc., Palo Alto, Calif., or other high throughput forms of biological data may be used as input data.

After identifying the data of interest (differentiated genes, in the current example), existing knowledge 104 may be reviewed, such as existing networks, for example, in an effort to identify pathways in the existing networks which are affected by the data of interest, thus establishing a user context 106. For example, analysis may be performed to identify pathways which are regulated by the differentiated genes which have been identified.

Alternatively, or additionally, a list of ALFA (Local Format Architecture) objects (resultant from converting data to the local format) created from the scientific literature, other textual data 112 or other data source such as diagrams 110 (for example, existing knowledge 104, such as existing diagrams, may be converted to ALFA objects, as shown), experimental data 102, or any other data 114, may be reviewed and compared with the data of interest to find associations therebetween, such information optionally be filtered according to user context 106. For example, a software tool known as BioFerret (Agilent Technologies, Inc., Palo Alto, Calif.) which is described in detail in co-pending, commonly owned application Ser. No. 10/033,823, filed Dec. 19, 2001 and titled “Domain-Specific Knowledge-based MetaSearch System and Methods of Using”, may be used for this application. Application Ser. No. 10/033,823 is incorporated herein, in its entirety, by reference thereto. However, a number of other means such as a keyword search of PubMed or other scientific database(s), for example, may be used to identify a corpus of relevant textual documents. The tools and methods disclosed in application Ser. No. 10/155,675 may be employed to extract knowledge from biological networks and convert the extractions to ALFA objects (local format). Further, virtually any data source or relevant portions thereof may be converted to ALFA objects for this purpose, as taught in application Ser. No. 10/154,524.

In this example, BioFerret was used, and referring to genes identified from the experimental data 102 as described above, one or more textual databases (e.g., Pubmed, or the like) were searched for textual documents containing the genes of interest. Sentences referring specifically to genes of interest were extracted and these genes and any interactions that the text described them being involved in were converted to ALFA objects.

One aspect of the present invention provides for extending an existing diagram by applying ALFA objects 108, such as those derived from the Bioferet processing described above, to an existing diagram. This may be accomplished, for example, by converting an existing diagram 104 to ALFA objects 108′ creating extending pathways 116 from the ALFA objects created by the Bioferret processing, and combining extending pathways 116 with the ALFA version of the existing diagram in locations where an extending pathway and the existing diagram share at least one common node or entity to form an extended or expanded diagram 118. An expansion operation may alternatively include using a filtering mechanism as described to remove one or more relations from a network. For example, where there are conflicting relations in a network, one such relation may be selected, while the one or more conflicting relations may be removed, or replaced by the selected relation, as a form of disambiguation by extension. Filters may be used to selectively expand a diagram, as discussed above. Diagrams may be newly created from ALFA objects. It is reiterated here, that these features are not limited to use of only ALFA objects converted from textual documents, as the ALFA objects used may be derived from any data source or any combination of the various types of data sources described.

A scoring mechanism is provided to score experimental data values against a diagram or expanded diagram to judge the level of significance of a pathway, relationship or entity, or any portion of a diagram in terms of the experimental data that is being considered.

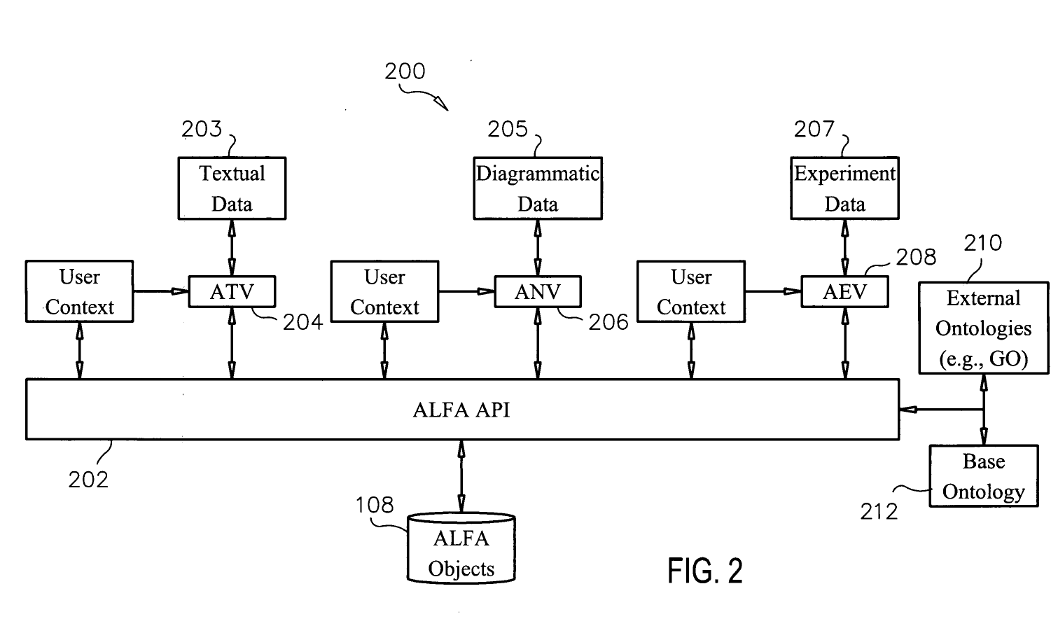

FIG. 2 schematically shows the components of an ALFA architecture 200 that may be employed in the formation of ALFA objects for use as described herein. An ALFA API (application programmer's interface) 202 is provided to allow users to access and use ALFA objects 108. ALFA API may be provided in JAVA code, for example. ALFA objects 108 are knowledge that is converted to a canonical or abstract representation. This abstract representation serves as a common language (local format) that can be used for textual representations, data representations and graphical representations of knowledge. This allows combining knowledge from different representations for comparison purposes, for constructing new and more detailed representations of knowledge, and the like.

While many different textual editors or viewers may be used to access textual representations of knowledge 203 and input such knowledge for conversion to the local format (some may also even data mine and automatically extract nouns and verbs, as noted above), textual viewer 204, as shown employs the textual viewer described in application Ser. No. 10/642,376, which provides for further user interaction for improvement of the knowledge gathered, as well as improvement of the accuracy when converting such knowledge to a local format.

A diagram viewer 206 may be used to view diagrammatic data 205 (e.g., biological diagrams), import graphical knowledge from the same and convert it to the local format (ALFA objects 108) for use with text and/or data. Further special features for conversion of biological diagrams, as well as construction of biological diagrams, which may be accompanied with use of the local format can be found in co-pending, commonly owned application Ser. No. 10/641,492.

Experimental data 207 may be imported and converted to ALFA objects 108, using a data viewer 208, for overlays on textual documents, biological diagrams, or incorporation of such knowledge with textual knowledge and/or graphical knowledge, through conversion of all types to a local format. An example of an experimental data viewer that may be used is described in application Ser. No. 10/403,762 or application Ser. No. 10/688,588.

External ontologies 210 such as gene ontology (GO) annotations, see http://www.geneontology.org, for example, may also be converted to ALFA objects for use in the present methods. Likewise, base ontology 212 may be converted to ALFA objects 108. Base ontology is a default, simple ontology that is provided along with ALFA. Base ontology comprises simple category names, such as proteins, genes, molecules, drugs, processes, etc., and their interrelationships as defined by the present inventors. Base ontology is currently represented as a text file that may be automatically read in by the ALFA API 202 whenever an ALFA file is read.

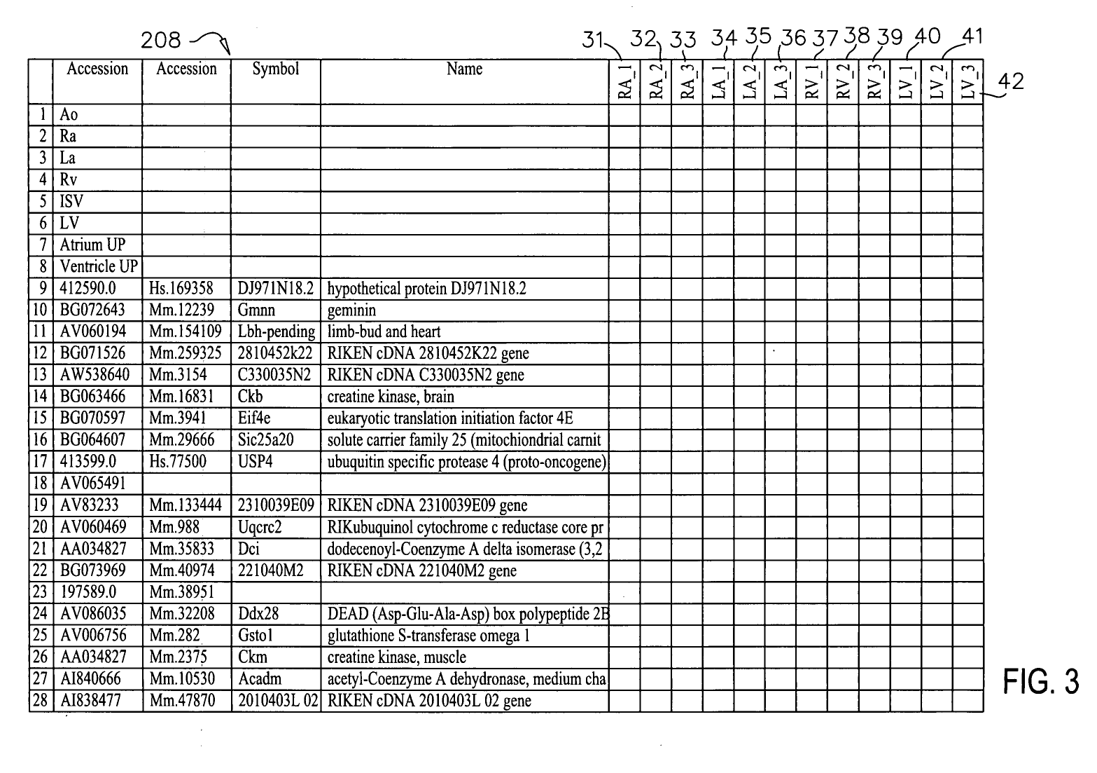

FIG. 3 shows a portion of a display from experimental viewer 208 being used to analyze twelve microarray experiments to study a particular phenomenon or disease, e.g. a particular type of cancer. In this example one set of experiments (experiments or columns 31-36) includes “normal” tissues (i.e., non-cancerous tissues and the other set (experiments 37-42) are cancer tissues that are run as experiments. By selecting on one of the rows of the matrix (each row represents expression values obtained for a particular gene or probe), a similarity sort of all the rows, based on the selected row was performed in the manner described in application Ser. No. 10/403,762. In this instance a very good separation of the two classes/sets was observed, as nearly all of the cells in the columns 31-36 as shown were color-coded green (meaning down-regulated) and nearly all of the cells in the columns 37-42 which are shown were color-coded red (which means up regulated). The interpretation of this result is that for the genes shown, the expression levels are highly differentiated when comparing the normal tissues to the cancerous tissues. From this it can be implied that the displayed genes may be involved in the regulation or in some way related to the cancer process that is going on in the cancer tissues.

Note however, that only twenty rows (i.e., twenty) are represented in the display of FIG. 3, purposes of meeting drawing requirements. It is not unusual for microarray experiments such as these to include twenty or thirty thousand genes/rows in total, and to identify one to three thousand rows that may be relevant to the phenomenon being studied. As such, the processing just described provides a quick and direct initial approach to identifying genes that may be involved in or related to a phenomenon being studied, to serve as a starting point for further analysis of the data such as finding pathways in which the related genes are involved, etc.

The list of genes (rows) identified by the process described with regard to FIG. 3 may be next inputted into a data mining tool such as Bioferret, which is described in application Ser. No. 10/033,823. Each of the genes is used in a query of one or more textual databases, such as PubMed, or the like, to search for scientific literature that mentions the identified genes. Queries may be based on gene or protein symbols, for example, which have been resolved using a biological information naming system (BNS) which is described in detail in co-pending, commonly owned application Ser. No. 10/154,529, filed May 22, 2002 and titled “Biotechnology Information Naming System”. Application Ser. No. 10/154,529 is hereby incorporated herein, in its entirety, by reference thereto. Alternatively, other unique identifiers of the data (e.g., other unique gene or protein identifiers or reference identifiers) may be used as bases for queries. This is a background process that typically isn't run in real time as it can be quite time consuming. For example, this processing may be done overnight for use the next morning. After identifying the scientific documents meeting the search requirements, sentences or portions of the documents that mention the genes of interest, as well as, potentially, relationships among genes, are extracted from the documents and converted to ALFA objects as described above. In this example, about three thousand seven hundred concepts (which represent genes, for example) and about ten thousand relations among these concepts were formed as ALFA objects.

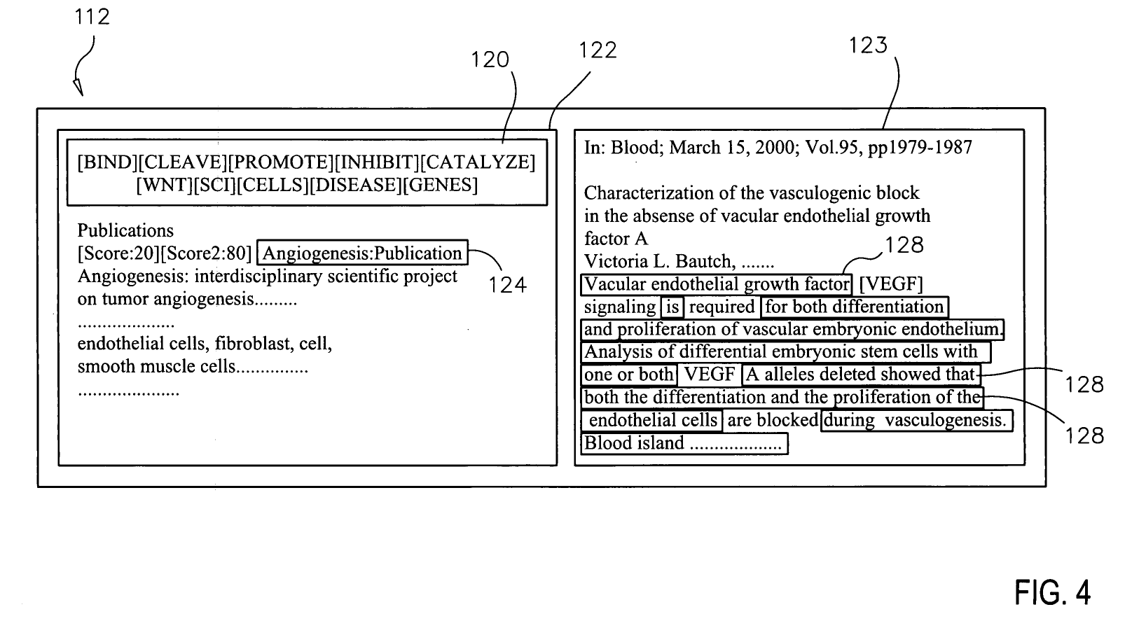

FIG. 4 is a schematic representation of a view displayed by a text viewer 204 during the process of extracting pertinent information from a scientific journal article that has been identified from a text database according to the techniques described above. The current list 120 of user context categories may be displayed, as shown. Text viewer 204 combines processes described in application Ser. No. 10/033,823 (BioFerret) with local user context processes described in co-pending, commonly owned application Ser. No. 10/155,304 filed May 22, 2002 and titled “System, Tools and Methods to Facilitate Identification and Organization of New Information Based on Context of User's Existing Information”. Application Ser. No. 10/155,304 is hereby incorporated herein, in its entirety, by reference thereto. For example, a set of genes in a microarray may be selected to serve as the user's context. Other examples of contexts include terms associated with the disease process, etc. Similarly, a set of verbs may be selected as context for interactions and relationships (referred to as interactions context). Each individual term in 120 is a pointer to a file, which contains a list of terms pertaining to that context. For example, “BIND” refers to a file bind.txt which may have verbs such as bind, combine, join, createbond, attach, etc. Any sentence in a piece of text that has at least one noun from the user context and one verb from the interactions context is then selected as an interesting sentence. An ALFA relation object is created from such a sentence marking all the identified nouns as concepts that are related. Thus, user context here is used to filter the potentially large set of sentences in the retrieved documents to identify specific relations.

A document or relevant section of a document may be selected from the list of identified documents displayed in viewer pane 122. In the example shown, “Angiogenesis: Publications” 124 has been selected by the user, which causes a detailed view of the article/publication to be displayed in viewer pane 126. Note the highlighted or blocked terms 128 that the viewer has identified to be used for conversion to ALFA objects. Using terms 128 and context terms as input, the system identifies nouns and verbs for matching the user contexts and selects sentences of interest (i.e., with at least one noun and verb matching that in the user context files). Those interesting sentences are then converted to ALFA objects characterizing biological concepts and relationships. This process may be performed automatically by the system or with user-guided input.

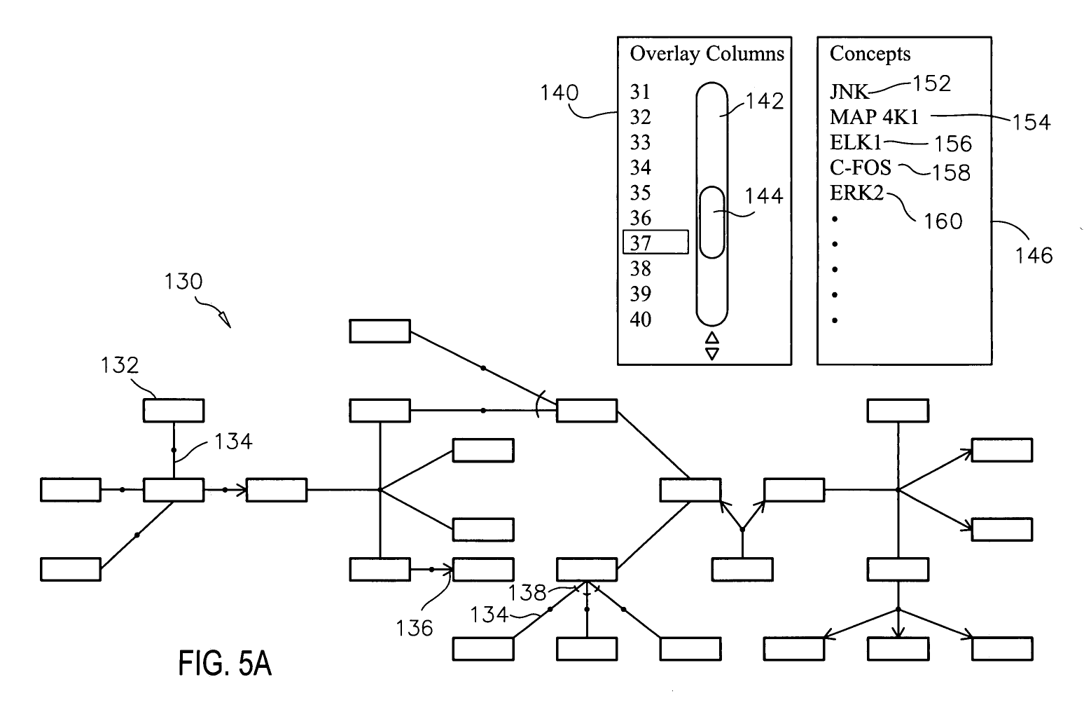

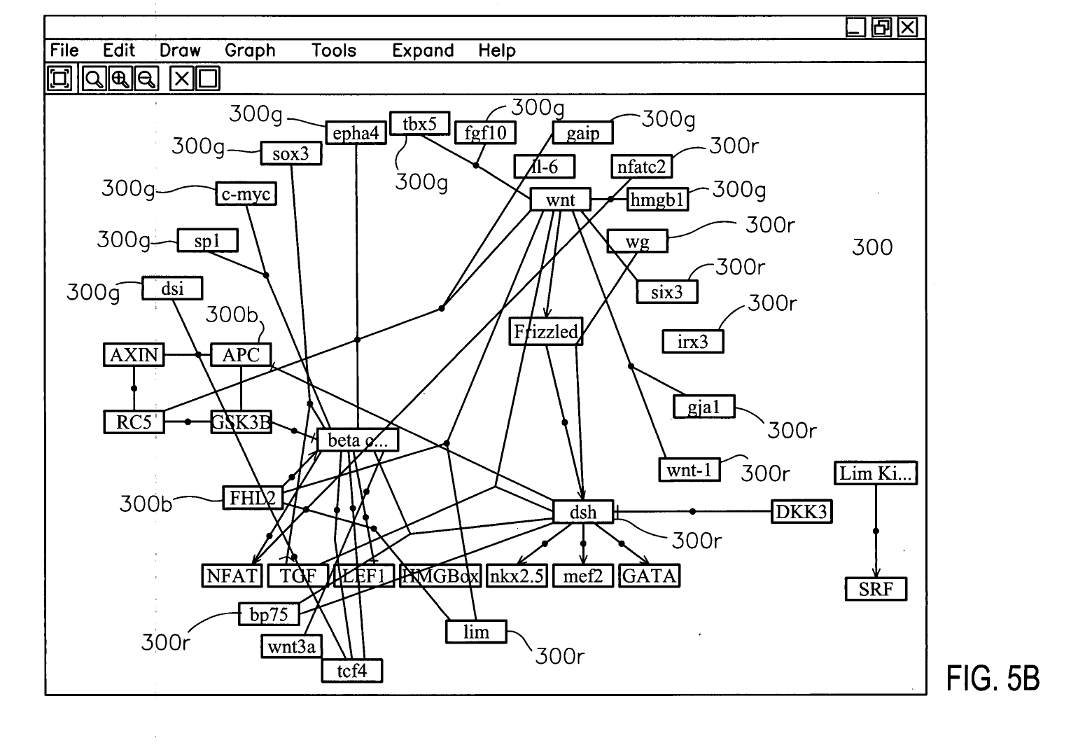

FIG. 5A is a schematic representation of a view displayed by a network viewer 206 displaying a network 130 of nodes (which, in this example, represent genes) and links (which in this example, represent relations between genes). An arrow-shaped icon 136 at the end of a link 134 represents an upregulation of the node that it points to, by the node from which the link 134 extends. A shield-shaped icon 138 at the end of a link 134 represents a downregulation or inhibition of the node that it is nearest, by the node from which the link 134 extends. In this example, viewer 206 is a network viewer of the type described in application Ser. No. 10/641,492. Viewer 206 is not only capable of importing, creating, and or modifying, as well as displaying network diagrams, but it is also capable of communicating with an experimental viewer 208, such as the experimental viewers described in application Ser. No. 10/403,762 or application Ser. No. 10/688,588, in order to overlay experimental data on the network diagram in the locations which represent the data being overlaid.

For example, an existing diagram, such as from a biological network database may be converted to the local format and displayed as network 130. In an example where viewer 206 communicates with an experimental data viewer 208 of the type described with respect to FIG. 3, a menu 140 of the columns 31-42 displayed in FIG. 1 may be displayed by viewer 206, as shown in FIG. 5. A scroll bar or other user selection tool may be provided to allow the user to choose which column from which to overlay the data on diagram 130. In the example shown, the user may select the desired column by clicking on the selector button 144 and sliding it either upwardly or downwardly to scroll up or down, respectively through the list of columns to the left of it. All the concepts present in the currently displayed network are contained within the concepts menu 146. e.g., JNK 152, MAP4K1 154, ELK1 156, c-FOS 158, ERK2 160, etc. When multiple networks are being examined, concepts menu 146 may be traversed and used as a networks menu. In this instance, concepts menu 146 is a larger table of contents menu, which can display multiple concepts (when a network is being viewed) or multiple networks. When a particular network is selected, that network is displayed in the viewer, with the concepts of the displayed network being displayed in concepts menu 146. When a concept is selected from concepts menu 146, that concept is highlighted on network 130 at all locations where it occurs in the displayed view of the network 130. In this way, concept menu 146 functions as a table of contents of the displayed network 130. Alternatively, or in addition thereto, the viewer may be provided with a simple tree-like structure (not shown) to represent networks and concepts that are currently being displayed.

Overlays may be performed on a network, as one example, to display the extracted knowledge as an overlay on top of existing networks, in a manner such as described in co-pending, commonly owned application Ser. No. 10/155,616 filed May 22, 2002 and titled “System and Methods for Visualizing Diverse Biological Relationships”, for example, or in application Ser. No. 10/642,376 or application Ser. No. 10/641,492. Application Ser. No. 10/155,616 is incorporated herein, in its entirety, by reference thereto. For example, correspondences and/or inconsistencies between multiple networks may be displayed by highlighting consistent and/or conflicting entities and relationships. Moreover, the invention allows for visual cues to discriminate the source for networks, entities, and relationships. For example, color-coding or other visual indicators may be used to identify that a particular overlay, such as a sub-network is information that was derived/extracted from a particular source, such as KEGG, or another different color or visual indicator may identify that a particular relationship that has been overlaid was extracted from scientific text, or from experimental data, etc.

When a network is expanded using multiple filters, the entities added to the network via different filters may be differentially overlaid, for example via differential color coding of their diagram nodes, to distinguish the contributions to the network expansion from each different filter. FIG. 5B shows an example of such differential overlays, carried out using a tool such as described in application Ser. No. 10/641,492. Network 300 displays a pathway (a network), which was extended using associations from the literature. The concepts corresponding to the associations are highlighted in red 300r and green 300g (the two colors signifying two different extensions). Concepts which are not extensions, but were part of the network 300 prior to performing the extensions are color-coded in a third color, such as grey 300b. Another mechanism uses differential coloring of the interaction edges in associations. In general, any network that consists of one or more associations may display these associations differently than the other pathway interactions (relationships).

Various techniques can be applied to visually differentiate a number of properties of a network. For example, a property that differentiates curated parts of a network from the non-curated parts, can be used to differentially display these parts of the network. Various techniques such as differential line widths, color coding, etc., can be employed to differentiate between curated and non-curated parts of a network. As another example, parts of the network that are part of a pathway versus those that are parts of an association can be differentially visualized. An “association” between a set of concepts is defined as an indirect link between these concepts. A “pathway interaction” is defined as one where there is a direct link between the concepts. For example, an experiment where an addition of a molecule leads to over-abundance of a particular protein shows an association between the added molecule and the over-abundant protein. However, the actual step-by-step interactions, if known, that lead to the over-abundance of the particular protein after addition of the molecule form the pathway interactions. Most interactions that are automatically inferred from the literature are in general associations.

The system also provides various tools for querying and traversing the network diagrams. The implementation of the local format by the present invention is a networked, graph data structure. Hence, many of the graph structure properties apply to the network diagrams. The provided features include: finding all occurrences of a given concept, finding all paths between two concepts, finding the shortest path between two concepts, finding the minimum spanning tree of the graph representing the network diagram, finding the size/order of the graph representing the network, finding connectivity distributions in the network, etc.

The system is also capable of identifying useful motifs or stencils. As described in co-pending, commonly assigned application Ser. No. ______ (application Ser. No. ______ not yet assigned, Attorney's Docket No. 10030635-1) filed Feb. 23, 2004 and titled “System, Tools and Method for Constructing Interactive Biological Diagrams”, describes stencils as visual motifs that represent commonly occurring structures in network diagrams, such as feed-forward loops, or multi-input relations, etc. application Ser. No. ______ (application Ser. No. ______ not yet assigned, Attorney's Docket No. 10030635-1) is hereby incorporated herein, in its entirety, by reference thereto. The present invention provides the capability to search for and identify stencils in a network diagram. Further, since stencils can have various rules associated with them, these can be used for various rule-checking operations while constructing and expanding networks. A rule is a procedure that can be run using data related to stencils, entities, and relationships. Rules can be declarative assertions that can be computationally verified, for example “the enzyme in this reaction must be a kinase”. Other examples of rules that can be associated with stencils include
Do the putative promotion/inhibition relationships in this interaction fit the values in the experimental data?
Does this biochemical reaction have the necessary components and preconditions?
Do the catalysts in this reaction exist in enough concentration to drive the reaction in a forward direction?

The system further provides tools for qualitative simulation of network models, providing features allowing a user to set the values of a set of nodes in the network, which are then processed within the network and propagated to show the effects downstream of the nodes having been inputted with set values. By performing this type of simulation on multiple networks, the networks may then be compared for downstream, comparing propagated values of the multiple networks for their consistency with experimental data (the experimental data are used to set the values of various concepts in the network), resolving inconsistencies in a network using experimental data (for example, experimental data can be used to validate, which of two mutually exclusive interactions occur between two concepts under a given experimental condition). Similarly, simulation is an equally effective technique for application to just one network, as comparisons for end actual cell states can be compared from experimental data, with simulated end cell states in a network. Thus a comparison between predicted values (from simulation overlays) may be made with actual values (from data overlays).

A typical use case of simulation may include setting the state of one or more molecules in a network model (such as active or inactive, to represent intervention by a drug compound being designed to alter the molecule's behavior) and propagating its effect downstream in the network. Potential behavior of the network under these different conditions (interference of various target molecules) can be computationally simulated to potentially identify the best drug targets against a particular disease.

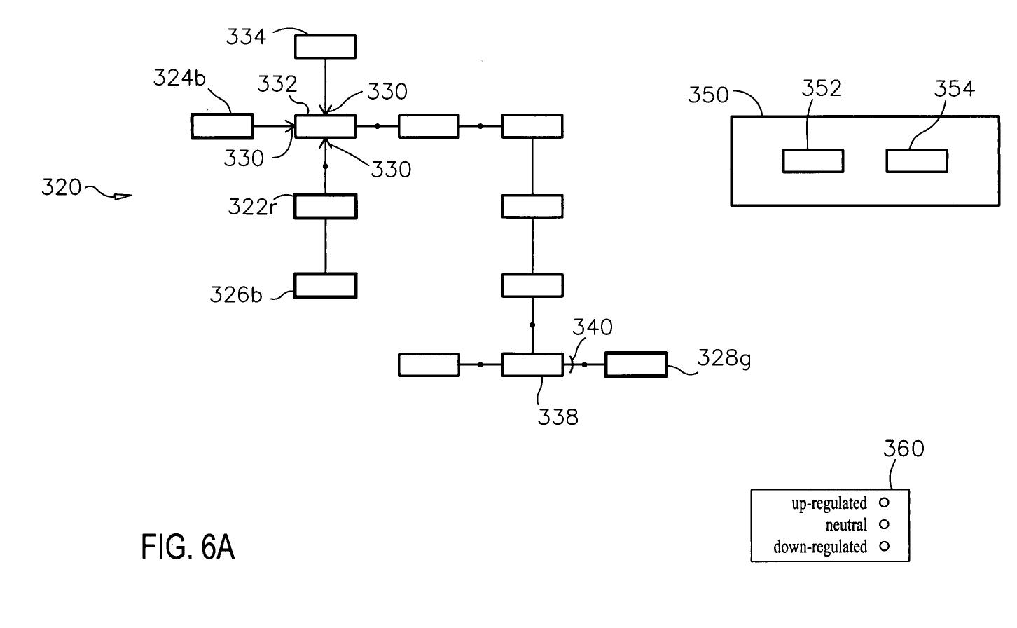

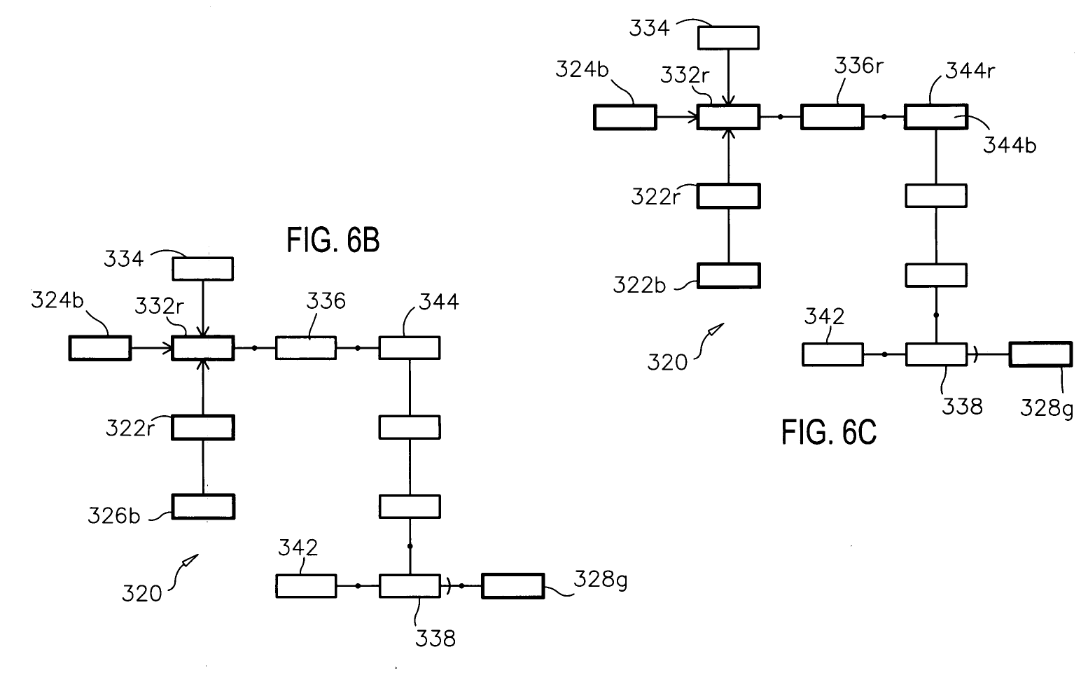

FIG. 6A is a simplified schematic representation of a portion of a network exemplifying simulation by the system. A column of experimental data has been overlaid from an experimental data viewer 208 with genes that match particular nodes displayed in diagram 320 according to the techniques described previously. In this example, the experimental data overlaid on node 322 is up-regulated and has been color-coded (outlined in red) 322r. The experimental data overlaid on node 328 is down-regulated and has been color-coded (outlined in green) 328g. The experimental data overlaid on nodes 324 and 326 is neutral and has been color-coded (outlined in black) 324b,326b. The remaining nodes displayed are not overlaid with experimental information.

The simulator tool is a rule-based tool provided with decision rules that may be plugged in and plugged out of the tool according to the user's needs. One of the rules employed in the visualization shown in FIG. 6A is that if a node is involved in a promotion relationship and if that node is up-regulated, then the node directly downstream of that node (i.e., at the other end of the promotion link) will be up-regulated as a result. Arrows 330 indicate that node 332 is indeed characterized by a promotion relationship between each of nodes 322, 324 and 334. According to the rule, if any one of nodes 322, 324 and 334 is up-regulated, then this should cause node 332 to be up-regulated. A pop-up menu, drop down menu, toolbar or other control mechanism 350 is provided to control the simulation activity of the tool. Among the functions provided, a control mechanism permits the user to step 352 through the simulation, or to propagate 354 the simulation fully through all of the downstream nodes.

By selecting the step 352 function, FIG. 6A shows that in fact, node 332 does become up-regulated and is color-coded (outlined in red) 332r. Even though it is unknown as to the activity level of node 334 and node 324b is neutral, the fact that node 322 is up-regulated is sufficient to up-regulate node 332 through the promotion relationship. By selecting step the next down stream nodes are processed in the simulation. In this example, node 336 would become up-regulated as one result of the next step.

Referring back to FIG. 6A it can be noted that node 328 is down regulated, and is involved in an inhibition relationship with node 338 as indicated by shield 340. When the simulation step described above is run, FIG. 6B show that node remains unchanged, since the down-regulation of node 328 is insufficient to down-regulate node 338 and no information is known about node 342. The simulation tool also provides input control means 360 which can be used to arbitrarily change or set the value of any selected node. This capability is useful for running “what if” simulation scenarios, such as the one described above where a node is blocked to mimic the action of a drug compound, after which the simulation can be run to study downstream effects on other nodes due to the blocking.

Although very simplistic rules have been described, the simulation tool may be provided with more complex rules, which may be modularly plugged into the tool as noted above. For example, with regard to the simulation described referencing FIGS. 6A and 6B, a rule which accounts for the accumulative effects of promoters may be substituted. Such as rule may consider the actual values of the data in nodes 322, 324 and 326, for example, and apply the cumulative effects to node 332 to determine whether the cumulative value meets a predefined threshold for up-regulating node 332. This is only one example of many rules and sets of rules which may be flexibly applied to the simulation tool to tailor it to the particular task at hand.

By propagating the simulation and thus finding expected values, states of nodes, another useful aspect of this tool allows additional experimental data to be overlaid on the diagram wherein actual experimental data values can be checked against expected or simulated values. For example, FIG. 6C shows the results of propagating the simulation through all of the displayed nodes. Although the expected value of node 344 is up-regulated as indicated by the red-colored outline 344r, the overlay of experimental data shows a discrepancy as the interior of node 344 is color-coded black indicating a neutral value 344b. This will alert the researcher that further study at this location is required to further modify diagram 320 or find some other explanation for the discrepancy. For example, changing directionality of one or more links in the diagram may improve its accuracy, or changing the meaning of the relationship between nodes may improve accuracy. Comparisons such as this may be performed on multiple diagrams and then the diagrams may be computationally compared as to the number and/or locations of discrepancies in each, as one technique for determining which is the best diagram for describing the experimental data. Further, portions of diagrams which are accurate may be combined with the present system to formulate a new diagram which may prove to be a more accurate predictor/explainer of the experimental data.

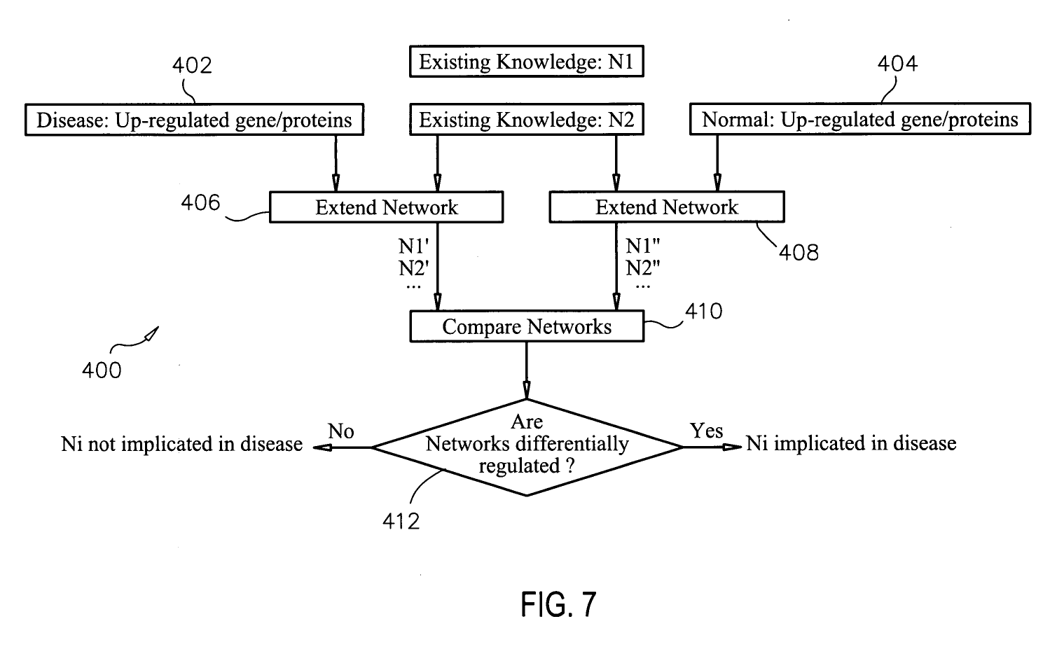

FIG. 7 is a flow chart 400 illustrating network extending capabilities as well as some of the processes for evaluating networks that may be carried out with the present system. Flow chart 400 is applied to the example described above where the high throughput experimental data being considered is microarray data for normal and diseased (e.g., cancerous) tissues. However, the principles discussed with regard to FIG. 6 may be applied generally to other types of high throughput data, including protein abundance data, mass spectrometry data, and other forms of high throughput biological data. Existing Knowledge N1, N2, . . . , Nn represents the various sources of existing data that were described above. The experimental values for the first class 402, in this case up regulated genes/proteins, may be identified in the manner described above with regard to FIG. 3, for example, with the similarity sort arranging the gene rows such that the top rows displayed those genes which were up regulated with regard to the diseased tissue samples, while down regulated with respect to the normal tissue samples.

The experimental values for the second class 404, are those values that show differentiation in an inverse manner to that shown by the values in the first class 402. For this example, the experimental values in the second class 404 include values for those rows of genes where the genes in the normal tissues are up regulated and the genes in the diseased tissues are down regulated. This class can be identified in the same way as discussed above with regard to the first class 402, and may be located at the bottom of the sort described with regard to FIG. 3, but not shown, since the display cannot accommodate all rows simultaneously in a manner where each row can be individually recognized with the eye.

Optionally, any networks contained in existing knowledge N1, N2, . . . , Nn may be extended with either of the experimental classes, 402,404, as shown at 406 and 408, respectively, according to any of the techniques described above. Further optionally, in the case where any particular network is extended/expanded using first class 402 at 406, and using second class 404 at 408, the resulting extended networks may then be compared at 410. One example of comparison is to simply visually compare the network as extended by the first class at 406 with the network as extended by the second class at 408. From such a visual comparison, a user may be able to readily notice difference in nodes (concepts) present, difference in structural properties of the networks compared, such as height, branching factor, etc., whether specific nodes are being differentially affected by both sets of expansions in the same way (assuming that the extended networks have unambiguous relations) or the like. For example, an expansion using first class 402 may show all promotions of a particular node, while an expansion using second class 404 may show all inhibitions of that same node. ALFA architecture is also configured for identifying such discrepancies automatically.

Still further, a user may recognize common sub-networks during a visual comparison, e.g., sub-networks from well-documented pathways in the sets of nodes and relations between them that can be visually inspected. Visual comparison may be sufficient to eliminate one extended version in cases where, for example, the user recognizes interactions that are not plausible in living systems, such as an interaction between two entities that are not know to interact in nature, for example, or an interaction that runs counter to well-established biochemical rules.

Another form of comparison 410 is a computational comparison that outputs the differences and similarities between the two extended networks.

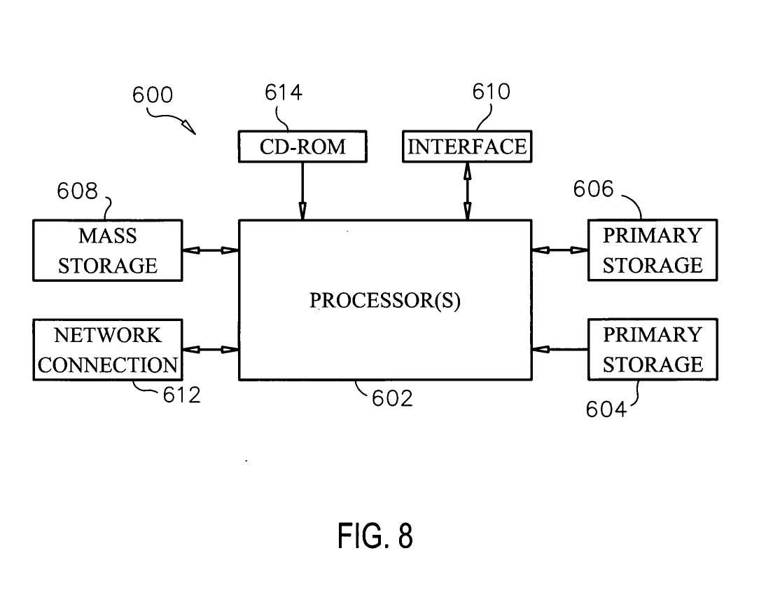

A tool for comparison of diagrams is provided in co-pending, commonly owned application Ser. No. 10/641,492. (see FIG. 8 and the description thereof in application Ser. No. 10/641,492). Using this tool, multiple network diagram overviews are displayed simultaneously in a spreadsheet viewer. The multiple network diagrams can thus be viewed simultaneously by the user for visual comparison. The view provided is a tabular viewer for browsing a collage/composite of multiple diagrams, allowing biological network information to be viewed in the context of many diagrams simultaneously, thereby increasing the probability of discovering properties among the various diagrams that would likely not have been noticed by viewing only a single diagram at a time. Additionally, the user can freely rearrange cells, rows, and columns in the table (each cell containing a different diagram, with the cells being arranged in rows and columns), positioning the diagrams in ways that accentuate similarities and correlations or draw attention to disparities. The basic operation of the spreadsheet viewer is as an overview navigation aid for multiple diagrams—clicking on one of the cells in the spreadsheet viewer results in a detailed view of the network diagram represented by that cell. A typical use may include (but is not restricted to this simple use case only) laying competing models in a grid and visually comparing (or computationally validating and visually displaying the consistencies and inconsistencies on each network diagram) these models against experimental data and identifying the best network.

Most biological models are approximate, general, and do not capture the nuances under different conditions. In fact, no one model may best describe the biological process under the given physical and experimental conditions. The spreadsheet view can be used to select different sub-networks from each of the networks (possibly generated from multiple alternative sources of information) displayed in the various cells, and combine these sub-networks such that the network constructed from the combination of these sub-networks is “optimally” validated by the experimental data or observations.

Additionally, or alternatively, each network, whether already having been extended or not, may be statistically assessed at 412 to determine whether the network being considered is differentially regulated in view of the experimental data being considered. Thus for example, a network derived from Existing Knowledge N1 can be considered at step 412 as to whether it is differentially regulated under the conditions identified by the experimental data in the first and second classes 402,404. Thus, the goal is to find networks that have a high Z-score in one class and a low Z-score in the other class. Additionally or alternatively, that same network as modified by the first class N1′ or as modified by the second class N1″ may be considered in the same manner. This is true for all of the networks derived from all of the existing sources of knowledge N1, N2, . . . , Nn whether singly or in combination.

The system statistically scores the network, whether it has been extended or not at 406,408, to give a measure of whether the network is significantly useful in describing the phenomenon that is being studied, when compared against the experimental data. As noted above, most analyses of high-throughput experimental data attempt to identify a subset of terms that are differentially expressed under different conditions. This subset may vary from tens to thousands of terms (genes in the case of a microarray experiment) and a method to identify interesting networks that contain an over- or under-abundance of these terms is very useful. The system may statistically analyze the relevance of any number of networks, created by employing one or more of the sources of data described above, and this analysis may also be applied to extended networks, as noted. For example, given a subset of differentially regulated genes from a microarray experiment and a list of networks (each represented in terms of its genes), a statistical score can be computed for each network in terms of over- or under-abundance of the presence of the genes from the subset that are also present in the network. The score can then be used to rank multiple networks in terms of their significance to the experimental data.

One means for scoring the networks, while not restricted to this specific mechanism, is discussed in Doniger et al., “MAPPFinder: using Gene Ontology and GenMAPP to create a global gene-expression profile from microarray data”. Genome Biology 2003, 4:R7, 2003, see also http://genomebiology.com/2003/4/1/R7. This means provides a criterion to identify and score statistical significance of networks as follows:

$$Z = \frac{r - np}{\sqrt{npq} \sqrt{1 - \frac{( n - 1 )}{( N - 1 )}}} \qquad ( 1 )$$

where
L=the list of terms measured in total. In the example described above, L is the list of genes measured by the microarray;
L1=the list of terms occurring in L which also occur in the network being statistically analyzed;
L2=the list of “interesting terms” selected from the total number L, i.e., those terms which pass some sort of discriminating criteria. In the example above, L2 may be the list of up-regulated diseased genes/proteins in 402 or the number of up-regulated normal genes/proteins in 404;
n=the number of terms in L1;
R=the number of terms in L2;
r=the number of terms in L1 and L2, e.g., in the example given, the number of genes in the intersection between L1 and L2;
N=the number of terms measured, i.e., the size of list L;
p=R/N; and
q=1−p.

Application Ser. No. ______ (application Ser. No. ______ not yet assigned, Attorney's Docket No. 10040171-1) filed concurrently herewith, and titled “Methods and System for Analyzing Term Frequency in Tabular Data” employs similar statistical methods for statistically analyzing the frequency of occurrence of word-based textual annotations associated with data. Application Ser. No. ______ (application Ser. No. ______ not yet assigned, Attorney's Docket No. 10040171-1) is hereby incorporated herein, in its entirety, by reference thereto.

A hypergeometric distribution of occurrence of a set of terms is assumed in the network being analyzed. The Z-score represents the statistical significance of seeing “r” terms in common between lists L1 and L2, given that only a set, L1, of terms was selected from the larger set of L terms. It represents a surprise in finding “r” terms when “n.p” terms are expected. A high positive (signifies statistical over-abundance) or negative value (signifies statistical under-abundance) of Z implies a significant surprise level, and hence, interestingness of the network based on the experimental data. The Z-scores values map to a substantially normal distribution. Typically, the current process considers an absolute value of the Z score greater than about three to indicate a three-sigma value, and is determined to be significantly differentiated. Therefore, in step 412, if the absolute value of the Z score is about 3 or greater, the system determines that the network being analyzed is significant, i.e., implicated in the phenomenon being studied, such as a disease process. It is emphasized that the system can analyze and statistically score any type of network, including existing networks from existing databases, an ALFA network created from literature or any source or combination of sources discussed above; an existing work extended by ALFA objects, manually drawn networks, curated networks, non-curated networks, partially curated networks, or any network that can be represented in ALFA format.

The system may be further employed to infer possible network models via the use of machine learning techniques, such as Bayesian belief networks. These techniques serve to predict causal relationships from observational and experimental data, such as gene expression data. The system may employ Bayesian and other machine learning techniques to generate a multiplicity of candidate networks/models, then apply a scoring metric to evaluate the candidate networks against the constraints imposed by the experimental data.

Bayesian inference is based on derivation of a model, M, from a corpus of data, D. From Bayes' theorem,
P(M|D)=P(M)*P(D|M)/P(D) (2)

The posterior, P(M|D), represents the probability that the model M is correct given the observed data, D. The prior, P(M), represents an estimate of the probability that model M is correct without having examined any data. P(D|M) represents the class-conditional density of the data, D, for a given model, M, and is experimentally determined from the training data. Thus, the posterior represents an updated belief in the probability that the model M is correct given the observed data and prior. The use of Bayesian inference and induction in bioinformatics is described in detail in Baldi, P. and Brunak, S., “Bioinformatics: The Machine Learning Approach”, MIT Press, 2001, which is incorporated herein, in its entirety, by reference thereto.

In methods that apply Bayesian inference to gene expression data, expression levels from individual genes are treated as variables and pairwise features of variables are examined. For example, if one can predict the expression level of a gene, X, by knowing the state of another gene, Y, independent of expression levels of the other genes, then it is probable that X and Y are co-regulated and it is possible that they are related in a biological interaction or process and that there is a pairwise causal relationship between Y and X. In the current invention, the probability that the products of two genes are functionally related can be predicted based upon the evidence presented, where the evidence presented may consist of a diverse range of information, such as evidence of co-regulation from gene expression data, evidence of functional relationship from protein-protein interaction databases, evidence of interactions derived from scientific literature, and explicit or implicit information provided by one or more users.

Once the probabilities of all pairwise interactions in a putative network mode are assessed, it is possible to stitch together a putative network model by applying a graph closure operation on the set of pairwise interactions. As a simplistic example, if there is a first interaction indicating that “A promotes B” and a second interaction indicating that “B promotes C”, then a sub-network describing “A promotes B promotes C” can be deduced. In this manner, a larger network can be built up from pairwise interactions and sub-networks. Moreover, the probability of the putative network model can be calculated as a weighted function of the pairwise interaction probabilities.

The analysis engine of this tool operates by generating a large number of candidate networks/models, then applying a scoring metric to evaluate the candidate networks against the constraints imposed by the experimental data. The use of priors is a strength of the Bayesian approach in that it allows incorporation of prior knowledge and constraints into the modeling process. Thus, modifying the priors can influence the scoring of different candidate networks/models. Examples of prior knowledge that may be employed in the scoring of candidate networks/models include: pairwise correlation and/or anti-correlation of gene expression profiles, which may strengthen the probability of a functional relationship between the products of those two genes; existence of an interaction between two gene products in a protein-protein interaction database, with the strength of that interaction influencing the probability of a functional relationship between those two gene products; citations in the literature indicating that an interaction exists for two or more gene products; and explicit or implicit indication from a user about the probability of an interaction between two or more gene products.

Often these tools are run iteratively. Each “run” generates an improved set of candidate networks. Modifying the prior knowledge following a “run” can influence the scoring of different candidate networks/models during a subsequent “run”. It is possible for a user, while exploring candidate networks in a visualization tool such as that described above, to provide relevance feedback to the analysis engine, thereby providing additional knowledge that can be used to optimize the next run of the engine. For example, the user can identify examples of “good” and “bad” candidate networks, which in turn may be provided to a Bayesian inference tool. The inference tool can use these examples to direct its search towards or away from certain candidate solutions, e.g. by weighting its scoring metrics in a way that candidate solutions that contain similarities to the marked networks are scored higher (for networks marked “good”) or lower (for networks marked “bad”). When the user is exploring candidate networks, her actions explicitly and/or implicitly build up “context” files, which are used by the inference tool during a subsequent “run”. There are a number of ways in which the user can explicitly provide context while exploring the candidate networks, for example by “lasso”-ing subnets and indicating with mouse gesture whether the subnet is a “good” or “bad” example. There are also several ways in which a user's operations while exploring the candidate networks can implicitly provide context: for example, the act of annotating a candidate network can be seen as an implication that the candidate network is of interest, thus a possibly “good” example. Also, the system may generate a context file to relatively score a network as good or bad, or provide input to make such determination based upon 60. The method of claim 57, wherein context files are produced based upon the number of times a network is accessed by a user, for example, or the length of time that a user uses a network, etc.

Biological processes are very complex and seldom act in isolation. However, traditional models are described in terms of small-scale and isolated network diagrams (such as KEGG pathways, for example). Biologists are now interested in identifying cross-talk between these familiar networks (or pathways) based on their experimental data and observations. The present system provides tools for querying multiple networks for common elements and displaying potential cross-talk (i.e., occurrence of the same concepts in multiple networks) among different networks. Using these tools, a user can query for all networks existing in the system that are affected if a certain bio-molecule (for example, a drug target) is altered. This may be particularly useful in conjunction with the simulator tool. For the drug simulation example, where the value of one or more nodes is changed and then the simulator is run to observe the effects downstream of the blocking of the one or more nodes in the diagram upon which the simulation is run, the user can next examine those downstream nodes which are affected and run a query for each of the affected nodes to identify other diagrams that include one or more of the affected nodes. Identification of such diagrams, i.e., identifying the cross-talk, will potentially lead to unexpected effects in other pathways that are caused by the drug.

FIG. 8 illustrates a typical computer system which may be employed in carrying out the present invention. The computer system 600 may include any number of processors 602 (also referred to as central processing units, or CPUs) that are coupled to storage devices including primary storage 606 (typically a random access memory, or RAM), primary storage 604 (typically a read only memory, or ROM). As is well known in the art, primary storage 604 acts to transfer data and instructions uni-directionally to the CPU and primary storage 606 is used typically to transfer data and instructions in a bi-directional manner Both of these primary storage devices may include any suitable computer-readable media such as those described above. A mass storage device 608 is also coupled bi-directionally to CPU 602 and provides additional data storage capacity and may include any of the computer-readable media described above. Mass storage device 608 may be used to store programs, data and the like and is typically a secondary storage medium such as a hard disk that is slower than primary storage. It will be appreciated that the information retained within the mass storage device 608, may, in appropriate cases, be incorporated in standard fashion as part of primary storage 606 as virtual memory. A specific mass storage device such as a CD-ROM 614 may also pass data uni-directionally to the CPU.

CPU 602 is also coupled to an interface 610 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to a computer or telecommunications network using a network connection as shown generally at 612. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for converting data types to the local format may be stored on mass storage device 608 or 614 and executed on CPU 608 in conjunction with primary memory 606, and one or more interfaces 610 (e.g., video displays) may be employed in displaying the viewer operations discussed herein.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, software, hardware, process, process step or steps, to the objective, spirit and scope of the present invention. All such modifications are intended to be within the scope of the claims appended hereto.

1. A method of extending a biological network, said method comprising the steps of:
providing at least a portion of the biological network in an interactive format representing concepts and relationships between concepts that occur in the biological network;
providing concepts and at least one relationship extracted from at least one data source in the interactive format, wherein the extractions are external to the biological network;
setting at least one filter to include at least one selected concept or relationship from the interactive format representation of the biological network;
matching at least one selected concept or relationship from the selected concepts and relationships contained in the filters with concepts and relationships provided in the interactive format from the at least one external data source; and
extending the biological network by merging concepts represented in the interactive format from the at least one data source matching concepts in the biological network.

2. The method of claim 1, further comprising assigning a level value to at least one of said at least one filters, wherein said level value determines the number of interactions and concepts by which an extracted concept is extended during said extending by merging the matching concepts in the biological diagram.

3. The method of claim 1, further comprising assigning a concepts per relation value to at least one of said at least one filters, wherein said concepts per relation value determines a number of interactions and concepts by which an extracted concept is extended during said extending by merging the matching concepts in the biological diagram.

4. The method of claim 1, further comprising the step of displaying the extended biological network.

5. The method of claim 1, wherein the at least one data source is selected from one or more of the group of data sources consisting of: textual data sources, experimental data sources, network diagram data sources, protein-protein interaction databases, manually constructed networks, and combinations thereof.

6. The method of claim 1, wherein multiple filters are set, and wherein the biological network is extended by only those interactions which have at least one matching concept selected in each of the multiple filters.

7. The method of claim 6, further comprising identifying the extended data in the biological network by an indicator representing the particular filter that the particular extension data was produced from.

8. The method of claim 1, wherein said concepts represent genes and said relationships represent interactions between genes.

9. The method of claim 1, wherein said filters are interactively set by a user.

10. The method of claim 1, further comprising displaying an identifier of the source of the data used to form each extended portion of the biological diagram.

11. The method of claim 1, further comprising, identifying at least one stencil corresponding to an extended portion of the biological network; and applying rules associated with the stencil to the extended portion to verify or disambiguate the extended portion.

12. A method comprising forwarding a result obtained from the method of claim 1 to a remote location.

13. A method comprising transmitting data representing a result obtained from the method of claim 1 to a remote location.

14. A method comprising receiving a result obtained from a method of claim 1 from a remote location.

15. A method of extending a biological network, said method comprising the steps of:
providing at least a portion of the biological network in an interactive format representing concepts and relationships between concepts that occur in the biological network;
constructing additional network diagrams from concepts and relationships extracted from at least one data source external to the biological network and converted to the interactive format;
interactively setting at least one filter to include at least one selected concept existing in the biological network;
searching the additional network diagrams to identify those additional network diagrams that contain at least one concept matching at least one selected concept; and
extending the biological network with all relationships from said additional network diagrams that are directly linked to at least one of the matching concepts, by merging the matching concepts on the biological network and extending the directly linked relationships, including all other concepts from the additional network diagrams that are directly linked by the directly linked relationships.

16. The method of claim 15, further comprising assigning a level value to at least one of said at least one filter, wherein said level value determines the number of interactions and concepts by which each selected concept is extended during said extending step.

17. The method of claim 15, further comprising assigning a concepts per relation value to at least one of said ate least one filter, wherein said concepts per relation value determines a number of interactions and concepts by which each selected concept is extended during said extending step.

18. The method of claim 15, further comprising the step of displaying the extended biological network.

19. The method of claim 15, wherein said concepts represent genes and said relationships represent interactions between genes, and wherein said at least one filter is interactively set by inputting a list of genes identified from experimental data and represented in the interactive format.

20. The method of claim 15, wherein said concepts represent proteins and said relationships represent interactions between proteins, and wherein said at least one filter is interactively set by inputting a list of proteins identified in a protein abundance list.

21. The method of claim 15, further comprising evaluating relevance of said extended network to representation of high throughput data.

22. The method of claim 21, wherein said evaluating comprises the steps of:
providing a set of data that are differentially expressed under different conditions;
searching the extended network to determine a number of matching data points to data points in said set of data; and
statistically analyzing the relevance of said extended network based on the number of data points that are in the set and also in the extended network, respectively.

23. The method of claim 22, wherein the set of data is a subset of a larger set of high throughput data that has been determined to be more differentially expressed than the remainder of the set of high throughput data.

24. The method of claim 23, wherein the larger set of high throughput data is gene expression data from at least one microarray experiment, and the subset characterizes genes from the data which are most differentially expressed under said different conditions.

25. The method of claim 23, wherein said statistically analyzing comprises scoring each network according to the following:
$$Z = \frac{r - np}{\sqrt{npq} \sqrt{1 - \frac{( n - 1 )}{( N - 1 )}}} \qquad ( 1 )$$
where
L=a list of terms in the larger set of high throughput data;
L1=a list of terms occurring in L which also occur in the network being statistically analyzed;
L2=a list of terms in the subset;
n=the number of terms in L1;
R=the number of terms in L2;
r=the number of terms common to both L1 and L2;
N=the number of terms in L;
p=R/N; and
q=1−p.

26. The method of claim 25, wherein a network is considered differentially regulated by the data from the subset when the absolute value of Z is greater than about 3.

27. A method of interactively manipulating biological data via user guidance, said method comprising the steps of:
providing a plurality of network diagrams represented in an interactive format representing concepts and relationships between concepts that occur in the network, wherein the concepts and relationships in each network diagram represent data from at least one of the data sources selected from the group consisting of textual data sources, experimental data sources, network diagram data sources, protein-protein interaction databases, manually constructed networks or any combination thereof, at least one of said network diagrams having been extended using data extracted from one of said data sources; and
evaluating said network diagrams by comparing said concepts and relationships among the network diagrams.

28. The method of claim 27, wherein said comparing comprises displaying at least a portion of said plurality of network diagrams simultaneously in a viewer to provide a direct visual comparison of the displayed network diagrams.

29. The method of claim 28, further comprising rearranging an order of the displayed network diagrams to display those diagrams which have similarities, disparities of common concepts and relationships, nearer or adjacent to one another.

30. The method of claim 28, wherein said evaluating comprises computationally validating and displaying at least one of consistencies and inconsistencies in the network diagrams.

31. The method of claim 28, wherein said evaluating comprises overlaying data from at least one data source and which is not represented by the network diagrams, over at least one of the network diagrams and indicating whether the overlaid data is consistent or inconsistent with the representation in the at least one network diagram that has been overlaid.

32. The method of claim 31, further comprising displaying an identifier of the source of the data overlaid on the at least one network diagram.

33. The method of claim 28, further comprising displaying an identifier of the source of the data used to form the extended portion of the at least one network diagram.

34. The method of claim 31, further comprising selecting the network diagram which best corresponds to the overlaid data.

35. The method of claim 34, wherein the selected network diagram has the greatest number of consistencies, least number of inconsistencies, or best score based on a combination of numbers of consistencies and inconsistencies.

36. The method of claim 31, further comprising selecting portions of at least two of the overlaid network diagrams, said portions having been overlaid with consistent data; and combining the consistent portions to form a new network diagram that is more consistent with the overlaid data than any of the other network diagrams.

37. The method of claim 28, further comprising differentiating, by visual display, curated portions of the network diagrams from non-curated portions of the network diagrams.

38. The method of claim 28, further comprising differentiating, by visual representation, associations from pathway interactions in the network diagrams.

39. The method of claim 27, wherein the interactive format is the local format.

40. The method of claim 27, further comprising:
selecting at least one concept;
searching all of the network diagrams for the at least one concept; and
identifying all occurrences of the selected at least one concept in all of the network diagrams and the locations of said occurrences.

41. The method of claim 27, further comprising:
selecting two concepts; and
searching all of the network diagrams to identify occurrences of the two concepts in each said diagram, identifying shortest paths between said two concepts and the locations of said paths.

42. The method of claim 27, further comprising:
selecting two concepts; and
searching all of the network diagrams to identify occurrences of the two concepts in each said diagram, identifying shortest paths between said two concepts in the respective diagrams, and determining the shortest path among the identified shortest paths.

43. The method of claim 27, further comprising calculating a minimum spanning tree of at least one of the network diagrams.

44. The method of claim 27, further comprising;
selecting at least one stencil; and
searching all of the network diagrams to identify occurrences of the at least one stencil and the locations of said occurrences.

45. A method for analyzing biological processes in a network diagram via simulation, said method comprising:
providing at least one biological network in an interactive format representing concepts and relationships between concepts that occur in the biological network;
setting a value of at least one concept in the biological network;
propagating a simulation by at least one relationship downstream of the at least one value set concept; and
displaying any effects on any concepts connected by the at least one relationship downstream of the at least one value set concept.

46. The method of claim 45, wherein the value for said setting a value step is applied from a value in experimental data corresponding to the concept for which each value is set, respectively.

47. The method of claim 45, wherein the value for said setting a value step is interactively set by a user.

48. The method of claim 45, further comprising comparing a value of at least one of the concepts downstream of a value set concept, resultant from said propagating, with a value in the experimental data corresponding to the at least one concept downstream, to validate a portion of the network, based on the experimental data or to identify a discrepancy between the portion of the network and the experimental data.

49. The method of claim 45, comprising providing multiple networks represented in the interactive format representing concepts and relationships between concepts that occur in the network, respectively, wherein said setting a value comprises setting the same value for the same concept in each corresponding network; and further comprising comparing any effects on any concepts connected by the at least one relationship downstream of the at least one value set concept, between each diagram to identify consistency or discrepancies between the networks.

50. The method of claim 49, wherein the value for each corresponding concept is applied from a value in experimental data corresponding to the concept for which each value is set, respectively.

51. The method of claim 49, wherein the value for each corresponding concept is interactively set by a user.

52. The method of claim 49, further comprising comparing a value of at least one of the concepts downstream of a value set concept, with respect to each diagram, resultant from said propagating, with a value in the experimental data corresponding to the at least one concept downstream, to validate a portion of the network, based on the experimental data or to identify a discrepancy between the portion of the network and the experimental data, respectively.

53. The method of claim 52, further comprising selecting the network diagram which contains the least number of discrepancies with respect to the experimental data, as the best model for the experimental data.

54. The method of claim 52, wherein values are set for a plurality of concepts in each network, to evaluate multiple portions of each network diagram.

55. The method of claim 54, further comprising selecting portions of at least two of the network diagrams each having different portions which contain the least number of discrepancies with respect to the experimental data relative to corresponding diagram portions of all other network diagrams; and combining the selected portions to form a new network diagram that is more consistent with the experimental data than any of the other network diagrams.

56. The method of claim 45, wherein effects from said propagating are determined by rules contained by a simulation tool performing the propagation.

57. The method of claim 45, wherein said effects are expected values.

58. The method of claim 45, comprising providing multiple networks represented in the interactive format representing concepts and relationships between concepts that occur in the network, respectively, wherein said setting a value comprises setting the same value for the same concept in each corresponding network, wherein said propagating is performed through all downstream relationships in each respective network, said method further comprising identifying each network containing downstream concepts having changed values affected by the propagation.

59. The method of claim 58, further comprising identifying subnetworks defined by the downstream concepts having changed values affected by the propagation, and their locations within the respective networks.

60. The method of claim 45, comprising providing multiple networks represented in the interactive format representing concepts and relationships between concepts that occur in the network, respectively, wherein said setting a value comprises setting the same value for the same concept in each corresponding network, identifying each network containing downstream concepts having changed values affected by the propagation; querying a database containing an additional number of networks represented in the interactive format, identifying networks from the additional number which contain at least one of the downstream concepts affected by the propagation in at least one of the networks upon which the propagation was performed, and propagating the networks identified in the additional number from the at least one identified concepts through all downstream relationships in each respective identified network.

61. The method of claim 60, further comprising identifying each network from the additional number containing downstream concepts having changed values affected by the propagation.

62. The method of claim 61, further comprising identifying subnetworks defined by the downstream concepts having changed values affected by the propagation in the networks identified from the additional number of networks, and their locations within the respective networks.

63. A method of inferring putative network models from high throughput data, said method comprising the steps of:
providing a set of data that are differentially expressed under different conditions;
identifying putative pairwise biological interactions amongst items in the data;
statistically analyzing the probability of said interactions based upon the high throughput data and at least one additional information source;
consolidating said interactions into candidate networks;
statistically analyzing said candidate networks based upon determined probabilities of said interactions contained within said candidate networks;
displaying the candidate networks to a user;
receiving relevance feedback from the user; and
iteratively refining the inference of putative network models, based on the candidate networks and identification of putative biological interactions using the relevance feedback.

64. The method of claim 63, wherein the set of high throughput data comprises gene expression data from at least one microarray experiment.

65. The method of claim 63, wherein the set of high throughput data comprises differential protein expression data from at least one mass spectrometry experiment.

66. The method of claim 63, wherein said identification of pairwise interaction is based on at least one of correlation of expression patterns and anti-correlation of expression patterns.

67. The method of claim 63, wherein said statistically analyzing the probability is based upon a scoring mechanism that additively combines information from the at least one additional source with initial expression correlation measures.

68. The method of claim 67, wherein the additive combination is performed using a Bayesian belief network.

69. The method of claim 63, wherein said at least one additional source comprises a multiplicity of additional sources selected from the group consisting of: protein-protein interaction databases, citations in scientific literature indicated at least one pairwise interaction, relevance feedback from a user about the probability of at least one pairwise interaction, explicit feedback mechanisms, implicit feedback mechanisms, and combinations thereof.

70. The method of claim 69, wherein said explicit feedback mechanisms comprise user identifications of good and bad networks.

71. The method of claim 69, wherein said implicit feedback mechanisms are selected from the group consisting of: recordation of a user annotation of a candidate network into a context file, a count of a number of times that a user accesses a candidate network, a measure of a length of time that a candidate network is the focus of a user interface, and combinations thereof.

72. The method of claim 63, wherein said consolidating comprises graphical closure of pairwise interactions into said candidate networks.

73. The method of claim 63, wherein said statistically analyzing said candidate networks comprises computing a weighted sum of the probabilities of said pairwise interactions contained within said candidate networks.

74. A method of facilitating the analysis of a biological network, said method comprising the steps of:
providing at biological network containing at least one of curated concepts and relationships, and non-curated concepts and relationships; and
displaying said curated concepts and relationships in a manner differentiating the display of said non-curated concepts and relationships.

75. The method of claim 74, wherein said curated concepts and relationships are displayed with lines having a first width and said non-curated concepts and relationships are displayed with lines having a second width different from said first width.

76. The method of claim 74, wherein said curated concepts and relationships are color-coded with a first color, and said non-curated concepts and relationships are color-coded with a second color different from said first color.

77. A system for extending a biological network, comprising:
means for providing at least a portion of the biological network in an interactive format representing concepts and relationships between concepts that occur in the biological network;
means for providing concepts and at least one relationship extracted from at least one data source in the interactive format, wherein the at least one data source is external to the biological network;
means for setting at least one filter to include at least one selected concept or relationship from the interactive format representation of the biological network;
means for matching at least one selected concept or relationship from the selected concepts and relationships contained in the filters with concepts and relationships provided in the interactive format from the at least one external data source; and
means for extending the biological network by merging concepts represented in the interactive format from the at least one data source matching concepts in the biological network.

78. The system of claim 77, further comprising means for assigning a level value to at least one of said at least one filters, wherein said level value determines a number of interactions and concepts by which an extracted concept is extended during said extending by merging the matching concepts in the biological diagram.

79. The system of claim 77, further comprising means for assigning a concepts per relation value to at least one of said at least one filters, wherein said concepts per relation value determines a number of interactions and concepts by which an extracted concept is extended during said extending by merging the matching concepts in the biological diagram.

80. The system of claim 77, further comprising means for displaying the extended biological network.

81. The system of claim 77, wherein said means for providing concepts and at least one relationship extracted from at least one data source are selected from the group consisting of: means for providing concepts and at least one relationship extracted from textual data sources, means for providing concepts and at least one relationship extracted from experimental data sources, means for providing concepts and at least one relationship extracted from network diagram data sources, means for providing concepts and at least one relationship extracted from protein-protein interaction databases, means for providing concepts and at least one relationship extracted from manually constructed networks, and combinations thereof.

82. The system of claim 77, further comprising means for identifying the extended data in the biological network by an indicator representing the particular filter that the particular extension data was produced from.

83. The system of claim 77, wherein said concepts represent genes and said relationships represent interactions between genes.

84. The method of claim 77, wherein said means for setting at least one filter comprises means for interactively setting said at least one filter by a user.

85. The system of claim 77, further comprising means for displaying an identifier of the source of the data used to form each extended portion of the biological diagram.

86. The system of claim 77, further comprising means for identifying at least one stencil corresponding to an extended portion of the biological network; and means for applying rules associated with the stencil to the extended portion to verify or disambiguate the extended portion.

87. A system for extending a biological network, comprising:
means for providing at least a portion of the biological network in an interactive format representing concepts and relationships between concepts that occur in the biological network;
means for constructing additional network diagrams from concepts and relationships extracted from at least one data source external to the biological network and converted to the interactive format;
means for interactively setting at least one filter to include at least one selected concept existing in the biological network;
means for searching the additional network diagrams to identify those additional network diagrams that contain at least one concept matching at least one selected concept; and
means for extending the biological network with all relationships from said additional network diagrams that are directly linked to at least one of the matching concepts, by merging at matching concepts on the biological network and extending the directly linked relationships, including all other concepts from the additional network diagrams that are directly linked by the directly linked relationships.

88. The system of claim 87, further comprising means for determining a number of interactions and concepts by which each selected concept is extended during said extending step.

89. The system of claim 87, further comprising means for displaying the extended biological network.

90. The system claim 87, wherein said concepts represent genes and said relationships represent interactions between genes, and wherein said at least one filter is interactively set by inputting a list of genes identified from experimental data and represented in the interactive format.

91. The system of claim 87, wherein said concepts represent proteins and said relationships represent interactions between proteins, and wherein said at least one filter is interactively set by inputting a list of proteins identified in a protein abundance list.

92. A system for interactively manipulating biological data via user guidance, comprising:
means for providing a plurality of network diagrams represented in an interactive format representing concepts and relationships between concepts that occur in the network, wherein the concepts and relationships in each network diagram represent data from at least one of the data sources selected from the group consisting of textual data sources, experimental data sources, network diagram data sources, protein-protein interaction databases, manually constructed networks or any combination thereof;
means for extending said network diagrams; and
means for evaluating said network diagrams by comparing said concepts and relationships among the network diagrams.

93. The system of claim 92, further comprising means for simultaneously displaying more than one of said plurality of network diagrams in a viewer to provide a direct visual comparison of the displayed network diagrams.

94. The system of claim 93, further comprising means for rearranging an order of the displayed network diagrams to display those diagrams which have similarities, disparities of common concepts and relationships, nearer or adjacent to one another.

95. The system of claim 93, further comprising means for computationally validating and displaying at least one of consistencies and inconsistencies in the network diagrams.

96. The system of claim 93, comprises means for overlaying data from at least one data source and which is not represented by the network diagrams, over at least one of the network diagrams and indicating whether the overlaid data is consistent or inconsistent with the representation in the at least one network diagram that has been overlaid.

97. The system of claim 96, further comprising means for displaying an identifier of the source of the data overlaid on the at least one network diagram.

98. The system of claim 93, further comprising means for displaying an identifier of the source of the data used to form an extended portion of the at least one network diagram.

99. The system of claim 96, further comprising means for selecting the network diagram which best corresponds to the overlaid data.

100. The system of claim 99, wherein the selected network diagram has the greatest number of consistencies, least number of inconsistencies, or best score based on a combination of numbers of consistencies and inconsistencies.

101. The system of claim 96, further comprising means for selecting portions of at least two of the overlaid network diagrams, said portions having been overlaid with consistent data; and means for combining the consistent portions to form a new network diagram that is more consistent with the overlaid data than any of the other network diagrams.

102. The system of claim 93, further comprising means for differentiating, by visual display, curated portions of the network diagrams from non-curated portions of the network diagrams.

103. The method of claim 93, further comprising means for differentiating, by visual representation, associations from pathway interactions in the network diagrams.

104. The system of claim 92, wherein the interactive format is the local format.

105. The system of claim 92, further comprising:
means for selecting at least one concept;
means for searching all of the network diagrams for the at least one concept; and
means for identifying all occurrences of the selected at least one concept in all of the network diagrams and the locations of said occurrences.

106. The system of claim 92, further comprising:
means for selecting two concepts; and
means for searching all of the network diagrams to identify occurrences of the two concepts in each said diagram and identifying shortest paths between said two concepts and the locations of said paths.

107. The system of claim 92, further comprising:
means for selecting two concepts; and
means for searching all of the network diagrams to identify occurrences of the two concepts in each said diagram, identifying shortest paths between said two concepts in said network diagrams, respectively, and determining the shortest path among the identified shortest paths.

108. The system of claim 92, further comprising:
means for selecting at least one stencil; and
means for searching all of the network diagrams to identify occurrences of the at least one stencil and the locations of said occurrences.

109. A system for analyzing biological processes in a network diagram via simulation, comprising:
means for providing at least one biological network in an interactive format representing concepts and relationships between concepts that occur in the biological network;
a simulation tool comprising means for setting a value of at least one concept in the biological network and means for propagating a simulation by at least one relationship downstream of the at least one value set concept; means for displaying any effects on any concepts connected by the at least one relationship downstream of the at least one value set concept.

110. The system of claim 109, wherein the value for said setting a value is applied from a value in experimental data corresponding to the concept for which each value is set, respectively.

111. The system of claim 109, wherein the value for said setting a value is interactively set by a user.

112. The system of claim 110, further comprising means for comparing a value of at least one of the concepts downstream of a value set concept, resultant from said propagating, with a value in the experimental data corresponding to the at least one concept downstream, to validate a portion of the network, based on the experimental data or to identify a discrepancy between the portion of the network and the experimental data.

113. The system of claim 109, comprising means for providing multiple networks represented in the interactive format representing concepts and relationships between concepts that occur in the network, respectively, wherein said means for setting a value sets the same value for the same concept in each corresponding network; and further comprising means for comparing any effects on any concepts connected by the at least one relationship downstream of the at least one value set concept, between each diagram to identify consistency or discrepancies between the networks.

114. The system of claim 113, wherein the value for each corresponding concept is applied from a value in experimental data corresponding to the concept for which each value is set, respectively.

115. The system of claim 113, wherein the value for at least one of said concepts corresponding in said corresponding networks is interactively applied by a user.

116. The system of claim 113, further comprising means for comparing a value of at least one of the concepts downstream of a value set concept, with respect to each diagram, resultant from said propagating, with a value in the experimental data corresponding to the at least one concept downstream, to validate a portion of the network, based on the experimental data or to identify a discrepancy between the portion of the network and the experimental data, respectively.

117. The system of claim 116, further comprising means for selecting the network diagram which contains the least number of discrepancies with respect to the experimental data, as the best model for the experimental data.

118. The system of claim 117, wherein said means for setting sets values with regard to multiple concepts in each said diagram, said system further comprising means for selecting portions of at least two of the network diagrams each having different portions which contain the least number of discrepancies with respect to the experimental data relative to corresponding diagram portions of all other network diagrams; and means for combining the selected portions to form a new network diagram that is more consistent with the experimental data than any of the other network diagrams.

119. The system of claim 109, wherein said simulation tool includes means for applying rules during said propagating to determine said effects.

120. The system of claim 109, wherein said effects are expected values.

121. The system of claim 109, comprising means for identifying subnetworks defined by the downstream concepts having changed values affected by the propagation, and their locations within the respective networks.

122. The system of claim 109, further comprising means for identifying each network containing downstream concepts having changed values affected by the propagation; means for querying a database containing an additional number of networks represented in the interactive format, means for identifying networks from the additional number which contain at least one of the downstream concepts affected by the propagation in at least one of the networks upon which the propagation was performed, and means for propagating the networks identified in the additional number from the at least one identified concepts through all downstream relationships in each respective identified network.

123. A system for evaluating network relevance to representation of high throughput data, comprising:
means for providing a set of data that are differentially expressed under different conditions;
means for searching at least one network representative of the set of data to determine the a number of matching data points in each network; and
means for statistically analyzing the relevance of each network based on the number of data points that are in the set and also in the network, respectively.

124. The system of claim 123, wherein the set of data is a subset of a larger set of high throughput data that has been determined to be more differentially expressed than the remainder of the set of high throughput data.

125. The system of claim 123, wherein said means for statistically analyzing comprises means for Z-scoring the networks.

126. A system for inferring putative network models from high throughput data, comprising:
means for providing a set of data that are differentially expressed under different conditions;
means for identifying putative pairwise biological interactions amongst items in the data;
means for statistically analyzing the probability of said interactions based upon the high throughput data and at least one additional information source;
means for consolidating said interactions into candidate networks;
means for statistically analyzing said candidate networks based upon determined probabilities of said interactions contained within said candidate networks;
means for displaying the candidate networks to a user;
means for receiving relevance feedback from the user; and
means for iteratively refining the inference of putative network models, based on the candidate networks and identification of putative biological interactions using the relevance feedback.

127. The system of claim 126, wherein said statistically analyzing the probability of said interactions comprises a Bayesian belief network

128. A system for facilitating the analysis of a biological network, comprising:
means for providing at biological network containing at least one of curated concepts and relationships, and non-curated concepts and relationships; and
means for displaying said curated concepts and relationships in a manner differentiating the display of said non-curated concepts and relationships.

129. A computer readable medium carrying one or more sequences of instructions for extending a biological network, wherein execution of one or more sequences of instructions by one or more processors causes the one or more processors to perform the steps of:
providing at least a portion of the biological network in an interactive format representing concepts and relationships between concepts that occur in the biological network;
providing concepts and at least one relationship extracted from at least one data source in the interactive format, wherein the at least one data source is external to the biological network;
setting at least one filter to include at least one selected concept or relationship from the interactive format representation of the biological network;
matching at least one selected concept or relationship from the selected concepts and relationships contained in the filters with concepts and relationships provided in the interactive format from the at least one external data source; and
extending the biological network by merging concepts represented in the interactive format from the at least one data source matching concepts in the biological network.

130. A computer readable medium carrying one or more sequences of instructions for extending a biological network, wherein execution of one or more sequences of instructions by one or more processors causes the one or more processors to perform the steps of:
providing at least a portion of the biological network in an interactive format representing concepts and relationships between concepts that occur in the biological network;
constructing additional network diagrams from concepts and relationships extracted from at least one data source external to the biological network and converted to the interactive format;
interactively setting at least one filter to include at least one selected concept existing in the biological network;
searching the additional network diagrams to identify those additional network diagrams that contain at least one concept matching at least one selected concept; and
extending the biological network with all relationships from said additional network diagrams that are directly linked to at least one of the matching concepts, by merging at matching concepts on the biological network and extending the directly linked relationships, including all other concepts from the additional network diagrams that are directly linked by the directly linked relationships.

131. A computer readable medium carrying one or more sequences of instructions for interactively manipulating biological data via user guidance, wherein execution of one or more sequences of instructions by one or more processors causes the one or more processors to perform the steps of:
providing a plurality of network diagrams represented in an interactive format representing concepts and relationships between concepts that occur in the network, wherein the concepts and relationships in each network diagram represent data from at least one of the data sources selected from the group consisting of textual data sources, experimental data sources, network diagram data sources, protein-protein interaction databases, manually constructed networks or any combination thereof, at least one of said network diagrams having been extended using data extracted from one of said data sources; and
evaluating said network diagrams by comparing said concepts and relationships among the network diagrams.

132. A computer readable medium carrying one or more sequences of instructions for analyzing biological processes in a network diagram via simulation, wherein execution of one or more sequences of instructions by one or more processors causes the one or more processors to perform the steps of:
providing at least one biological network in an interactive format representing concepts and relationships between concepts that occur in the biological network;
setting a value of at least one concept in the biological network;
propagating a simulation by at least one relationship downstream of the at least one value set concept; and
displaying any effects on any concepts connected by the at least one relationship downstream of the at least one value set concept.

133. A computer readable medium carrying one or more sequences of instructions for inferring putative network models from high throughput data, wherein execution of one or more sequences of instructions by one or more processors causes the one or more processors to perform the steps of:
providing a set of data that are differentially expressed under different conditions;
identifying putative pairwise biological interactions amongst items in the data;
statistically analyzing the probability of said interactions based upon the high throughput data and at least one additional information source;
consolidating said interactions into candidate networks;
statistically analyzing said candidate networks based upon determined probabilities of said interactions contained within said candidate networks;
displaying the candidate networks to a user;
receiving relevance feedback from the user;
iteratively refining the inference of putative network models, based on the candidate networks and identification of putative biological interactions using the relevance feedback.
